SEQUENCE LISTING (28 sequences)

SEQ ID NO: 1 - DNA, 765 bp, Artificial Sequence
  synthetic P271 (P266) antimicrobial protein construct, P225 alternative construct, vector seq-his6-GP36 muralytic domain-RRR-BPI TMD-RRR, 6xHis tag - GP36 CD - RRR - BPI TMD - RRR
atgggccatcatcatcatcatcatggtgtagctcttgatcgcacgcgggttgatccccaggcagtcggcaacgaggtgctcaagcgcaacgcggataagctgaatgcgatgcggggcgccgagtacggtgccaacgtcaaggtcagcggcacggacattcgcatgaacgggggtaacagtgccggcatgctgaagcaggacgtgttcaactggcggaaggaactggctcagttcgaggcttaccgaggggaggcgtataaggatgccgatggttatagtgtgggcctggggcattacctgggcagtggcaatgctggggcaggtactacagtcacgcctgagcaagccgcgcagtggttcgccgaggacaccgaccgcgcactcgaccagggtgtgaggttggccgacgagctgggcgttacgaacaatgcctctatcctgggattggccggtatggccttccagatgggcgaaggacgtgcccggcagttccgtaacaccttccaggcgatcaaggatcgcaacaaggaagccttcgaggctggtgtgcgaaacagcaagtggtacacgcagacgcccaaccgggccgaggcattcatcaagcgcatggcgccccacttcgatacaccgagtcaaatcggtgtcgattggtacagcgccgcaacagcggagcgccgtcgcgcgtccctgatggtgctggtcgccataggcaccgccgtgacagcggccgtcaaccctggcgtcgtggtcaggcgccgtcgctga

SEQ ID NO: 2 - protein, 254 aa, Artificial Sequence
  synthetic P271 (P266) antimicrobial protein construct, P225 alternative construct, vector seq-his6-GP36 muralytic domain-RRR-BPI TMD-RRR, 6xHis tag - GP36 CD - RRR - BPI TMD - RRR
MGHHHHHHGVALDRTRVDPQAVGNEVLKRNADKLNAMRGAEYGANVKVSGTDIRMNGGNSAGMLKQDVFNWRKELAQFEAYRGEAYKDADGYSVGLGHYLGSGNAGAGTTVTPEQAAQWFAEDTDRALDQGVRLADELGVTNNASILGLAGMAFQMGEGRARQFRNTFQAIKDRNKEAFEAGVRNSKWYTQTPTGAEAFIKRMAPHFDTPSQIGVDWYSAATAERRRASLMVLVAIGTAVTAAVNPGVVVRRRR

SEQ ID NO: 3 - DNA, 765 bp, Artificial Sequence
  synthetic P275 construct, soluble P271 (P266) variant
atgggccatcatcatcatcatcatggtgtagctcttgatcgcacgcgggttgatccccaggcagtcggcaacgaggtgctcaagcgcaacgcggataagctgaatgcgatgcggggcgccgagtacggtgccaacgtcaaggtcagcggcacggacattcgcatgaacgggggtaacagtgccggcatgctgaagcaggacgtgttcaactggcggaaggaactggctcagttcgaggcttaccgaggggaggcgtataaggatgccgatggttatagtgtgggcctggggcattacctgggcagtggcaatgctggggcaggtactacagtcacgcctgagcaagccgcgcagtggttcgccgaggacaccgaccgcgcactcgaccagggtgtgaggttggccgacgagctgggcgttacgaacaatgcctctatcctgggattggccggtatggccttccagatgggcgaaggacgtgcccggcagttccgtaacaccttccaggcgatcaaggatcgcaacaaggaagccttcgaggctggtgtgcgaaacagcaagtggtacacgcagacgcccaaccgggccgaggcattcatcaagcgcatggcgccccacttcgatacaccgagtcaaatcggtgtcgattggtacagcgccgcaacagcggagcgccgtcgcgcgtccctgatggagctggacgccaaaggcaccgccgtgacagcggccgtcaaccctggcgtcgtggtcaggcgccgtcgctga

SEQ ID NO: 4 - protein, 254 aa, Artificial Sequence
  synthetic P275 construct, soluble P271 (P266) variant polypeptide
MGHHHHHHGVALDRTRVDPQAVGNEVLKRNADKLNAMRGAEYGANVKVSGTDIRMNGGNSAGMLKQDVFNWRKELAQFEAYRGEAYKDADGYSVGLGHYLGSGNAGAGTTVTPEQAAQWFAEDTDRALDQGVRLADELGVTNNASILGLAGMAFQMGEGRARQFRNTFQAIKDRNKEAFEAGVRNSKWYTQTPTGAEAFIKRMAPHFDTPSQIGVDWYSAATAERRRASLMELDAKGTAVTAAVNPGVVVRRRR

SEQ ID NO: 5 - protein, 267 aa, Artificial Sequence
  synthetic GP36 MD - P134 holin TMD construct polypeptide, GP36CD-P134holin fusion protein
MGHHHHHHHHHHSSGHIEGRHMGVALDRTRVDPQAVGNEVLKRNADKLNAMRGAEYGANVKVSGTDIRMNGGNSAGMLKQDVFNWRKELAQFEAYRGEAYKDADGYSVGLGHYLGSGNAGAGTTVTPEQAAQWFAEDTDRALDQGVRLADELGVTNNASILGLAGMAFQMGEGRARQFRNTFQAIKDRNKEAFEAGVRNSKWYTQTPTGAEAFIKRMAPHFDTPSQIGVDWYSAATAERRREIASLCAAVLTALYVGAQLITLLRRR

SEQ ID NO: 6 - protein, 274 aa, Artificial Sequence [sequence omitted]

SEQ ID NO: 7 - protein, 254 aa, Artificial Sequence
  synthetic P317 construct, soluble P271 (P266) variant polypeptide
MGHHHHHHGVALDRTRVDPQAVGNEVLKRNADKLNAMRGAEYGANVKVSGTDIRMNGGNSAGMLKQDVFNWRKELAQFEAYRGEAYKDADGYSVGLGHYLGSGNAGAGTTVTPEQAAQWFAEDTDRALDQGVRLADELGVTNNASILGLAGMAFQMGEGRARQFRNTFQAIKDRNKEAFEAGVRNSKWYTQTPTGAEAFIKRMAPHFDTPSQIGVDWYSAATAERRRASLMKLKAIGTAVTAAVNPGVVVRRRR

SEQ ID NO: 8 - protein, 146 aa, Homo sapiens
  mature human IL-13 polypeptide
MHPLLNPLLLALGLMALLLTTVIALTCLGGFASPGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGQFN

SEQ ID NO: 9 - protein, 146 aa, Artificial Sequence
  synthetic human IL-13 variant polypeptide
MHPDDNPDDDALGLMADDDTTVRALTCLGGFASPGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGQFN

SEQ ID NO: 10 - protein, 192 aa, Homo sapiens
  human BAX polypeptide
MDGSGEQPRGGGPTSSEQIMKTGALLLQGFIQDRAGRMGGEAPELALDPVPQDASTKKLSECLKRIGDELDSNMELQRMIAAVDTDSPREVFFRVAADMFSDGNFNWGRVVALFYFASKLVLKALCTKVPELIRTIMGWTLDFLRERLLGWIQDQGGWDGLLSYFGTPTWQTVTIFVAGVLTASLTIWKKMG

SEQ ID NO: 11 - protein, 192 aa, Artificial Sequence
  synthetic BAX variant polypeptide
MDGSGEQPRGGGPTSSEQIMKTGALLLQGFIQDRAGRMGGEAPELALDPVPQDASTKKLSECLKRIGDELDSNMELQRMIAAVDTDSPREVFFRVAADMFSDGNFNWGRVVALFYFASKLVLKALCTKVPELIRTIMGWTLDFLRERLLGWIQDQGGWDGLLSYFGTPTWQTDTDFDAGDDTASDTDWKKMG

SEQ ID NO: 12 - protein, 110 aa, Escherichia coli
  Escherichia coli serovar O104H4 strain C227-11 SecG polypeptide
MYEALLVVFLIVAIGLVGLIMLQQGKGADMGASFGAGASATLFGSSGSGNFMTRMTALLATLFFIISLVLGNINSNKTNKGSEWENLSAPAKTEQTQPAAPAKPTSDIPN

SEQ ID NO: 13 - protein, 110 aa, Artificial Sequence
  synthetic Escherichia coli serovar O104H4 strain C227-11 SecG variant polypeptide
MYEADDVVFLDVADGDVGDDMLQQGKGADMGASFGAGASATLFGSSGSGNFMTRMTADDATDFFDDSDVDGNDNSNKTNKGSEWENLSAPAKTEQTQPAAPAKPTSDIPN

SEQ ID NO: 14 - protein, 670 aa, Yarrowia lipolytica
  Yarrowia lipolytica CLIB122 Kar2p heat shock protein (BIP homolog)
MKFSMPSWGVVFYALLVCLLPFLSKAGVQADDVDSYGTVIGIDLGTTYSCVGVMKGGRVEILANDQGSRITPSYVAFTEDERLVGDAAKNQAANNPFNTIFDIKRLIGLKYKDESVQRDIKHFPYKVKNKDGKPVVVVETKGEKKTYTPEEISAMILTKMKDIAQDYLGKKVTHAVVTVPAYFNDAQRQATKDAGIIAGLNVLRIVNEPTAAAIAYGLDHTDDEKQIVVYDLGGGTFDVSLLSIESGVFEVLATAGDTHLGGEDFDYRVIKHFVKQYNKKHDVDITKNAKTIGKLKREVEKAKRTLSSQMSTRIEIESFFDGEDFSETLTRAKFEELNIDLFKRTLKPVEQVLKDSGVKKEDVHDIVLVGGSTRIPKVQELLEKFFDGKKASKGINPDEAVAYGAAVQAGVLSGEDGVEDIVLLDVNPLTLGIETTGGVMTKLINRNTNIPTKKSQIFSTAVDNQSTVLIQVFEGERTMSKDNNLLGKFELKGIPPAPRGVPQIEVTFELDANGILRVTAHDKGTGKSETITITNDKGRLSKDEIERMVEEAERFAEEDALIRETIEAKNSLENYAHSLRNQVADKSGLGGKISADDKEALNDAVTETLEWLEANSVSATKEDFEEKKEALSAIAYPITSKIYEGGEGGDESNDGGFYADDDEAPFHDEL

SEQ ID NO: 15 - protein, 670 aa, Artificial Sequence
  synthetic Yarrowia lipolytica CLIB122 Kar2p heat shock protein (BIP homolog) variant polypeptide
MKFSMPSWGDDFYADDDCDDPFDSKAGVQADDVDSYGTVIGIDLGTTYSCVGVMKGGRVEILANDQGSRITPSYVAFTEDERLVGDAAKNQAANNPFNTIFDIKRLIGLKYKDESVQRDIKHFPYKVKNKDGKPVVVVETKGEKKTYTPEEISAMILTKMKDIAQDYLGKKVTHAVVTVPAYFNDAQRQATKDAGIIAGLNVLRIVNEPTAAAIAYGLDHTDDEKQIVVYDLGGGTFDVSLLSIESGVFEVLATAGDTHLGGEDFDYRVIKHFVKQYNKKHDVDITKNAKTIGKLKREVEKAKRTLSSQMSTRIEIESFFDGEDFSETLTRAKFEELNIDLFKRTLKPVEQVLKDSGVKKEDVHDIVLVGGSTRIPKVQELLEKFFDGKKASKGINPDEAVAYGAAVQAGVLSGEDGVEDIVLLDVNPLTLGIETTGGVMTKLINRNTNIPTKKSQIFSTAVDNQSTVLIQVFEGERTMSKDNNLLGKFELKGIPPAPRGVPQIEVTFELDANGILRVTAHDKGTGKSETITITNDKGRLSKDEIERMVEEAERFAEEDALIRETIEAKNSLENYAHSLRNQVADKSGLGGKISADDKEALNDAVTETLEWLEANSVSATKEDFEEKKEALSAIAYPITSKIYEGGEGGDESNDGGFYADDDEAPFHDEL

SEQ ID NO: 16 - protein, 173 aa, Homo sapiens
  human cathelecidin CAP18 polypeptide
MGTMKTQRDGHSLGRWSLVLLLLGLVMPLAIIAQVLSYKEAVLRAIDGINQRSSDANLYRLLDLDPRPTMDGDPDTPKPVSFTVKETVCPRTTQQSPEDCDFKKDGLVKRCMGTVTLNQARGSFDISCDKDNKRFALLGDFFRKSKEKIGKEFKRIVQRIKDFLRNLVPRTES

SEQ ID NO: 17 - protein, 173 aa, Artificial Sequence
  synthetic human cathelecidin CAP18 variant polypeptide
MGTMKTQRDGHSLGRWSLVLRRRGLVMPLAKKAQVLSYKEAVLRAIDGINQRSSDANLYRLLDLDPRPTMDGDPDTPKPVSFTVKETVCPRTTQQSPEDCDFKKDGLVKRCMGTVTLNQARGSFDISCDKDNKRFALLGDFFRKSKEKIGKEFKRIVQRIKDFLRNLVPRTES

SEQ ID NO: 18 - protein, 207 aa, Enterobacteria phage PRD1
  Enterobacteria phage PRD1 DNA delivery protein
MEKVKAWLIKYKWWIVAAIGGLAAFLLLKNRGGGSGGGGEYMVGSGPVYQQAGSGAVDNTMALAALQANTQLSAQNAQLQAQMDASRLQLETQLNIETLAADNAHYSTQSQLQLGMAQVDLSKYLGDLQSTTSTALAGMQSDTAKYQSNIQLQAENIRANTSLAEIDAQKYIVGKQADIAKYQAKTERRGQDYGFALGLLNFGGKFF

SEQ ID NO: 19 - protein, 207 aa, Artificial Sequence
  synthetic Enterobacteria phage PRD1 DNA delivery protein variant
MEKVKAWLIKYKWWIVAADGGLAAFKRRKNRGGGSGGGGEYMVGSGPVYQQAGSGAVDNTMALAALQANTQLSAQNAQLQAQMDASRLQLETQLNIETLAADNAHYSTQSQLQLGMAQVDLSKYLGDLQSTTSTALAGMQSDTAKYQSNIQLQAENIRANTSLAEIDAQKYIVGKQADIAKYQAKTERRGQDYGFALGLLNFGGKFF

SEQ ID NO: 20 - protein, 265 aa, Enterobacteria phage PRD1
  Enterobacteria phage PRD1 transglycosylase P7
MSGALQWWETIGAASAQYNLDPRLVAGVVQTESSGNPRTTSGVGAMGLMQLMPATAKSLGVTNAYDPTQNIYGGAALLRENLDRYGDVNTALLAYHGGTNQANWGAKTKSYPGKVMKNINLLFGNSGPVVTPAAGIAPVSGAQEMTAVNISDYTAPDLTGLTMGAGSPDFTGGASGSWGEENIPWYRVDKHVANAAGSAYDAVTDAVSAPVEAAGNYALRGVVIIAAVAIVVVGLYFLFQDEINSAAMKMIPAGKAAGAAAKALA

SEQ ID NO: 21 - protein, 265 aa, Artificial Sequence
  synthetic Enterobacteria phage PRD1 transglycosylase P7 variant protein
MSGALQWWETIGAASAQYNLDPRLVAGVVQTESSGNPRTTSGVGAMGLMQLMPATAKSLGVTNAYDPTQNIYGGAALLRENLDRYGDVNTALLAYHGGTNQANWGAKTKSYPGKVMKNINLLFGNSGPVVTPAAGIAPVSGAQEMTAVNISDYTAPDLTGLTMGAGSPDFTGGASGSWGEENIPWYRVDKHVANAAGSAYDAVTDAVSAPVEAAGNYALRGVDKRAAVAIKRKGLYFLFQDEINSAAMKMIPAGKAAGAAAKALA

SEQ ID NO: 22 - protein, 321 aa, Escherichia coli
  Escherichia coli K-12 colicin N, chain A
HGDNNSKPKPGGNSGNRGNNGDGASAKVGEITITPDNSKPGRYISSNPEYSLLAKLIDAESIKGTEVYTFHTRKGQYVKVTVPDSNIDKMRVDYVNWKGPKYNNKLVKRFVSQFLLFRKEEKEKNEKEALLKASELVSGMGDKLGEYLGVKYKNVAKEVANDIKNFHGRNIRSYNEAMASLNKVLANPKMKVNKSDKDAIVNAWKQVNAKDMANKIGNLGKAFKVADLAIKVEKIREKSIEGYNTGNWGPLLLEVESWIIGGVVAGVAISLFGAVLSFLPISGLAVTALGVIGIMTISYLSSFIDANRVSNINNIISSVIR

SEQ ID NO: 23 - protein, 321 aa, Artificial Sequence
  synthetic Escherichia coli K-12 colicin N, chain A variant polypeptide
HGDNNSKPKPGGNSGNRGNNGDGASAKVGEITITPDNSKPGRYISSNPEYSLLAKLIDAESIKGTEVYTFHTRKGQYVKVTVPDSNIDKMRVDYVNWKGPKYNNKLVKRFVSQFLLFRKEEKEKNEKEALLKASELVSGMGDKLGEYLGVKYKNVAKEVANDIKNFHGRNIRSYNEAMASLNKVLANPKMKVNKSDKDAIVNAWKQVNAKDMANKIGNLGKAFKVADLAIKVEKIREKSIEGYNTGNWGPLLLEVESWIIGGRKAGDAESLFGARLSFLPKSGLAKTALGKRGRMTISYLSSFIDANRVSNINNIISSVIR

SEQ ID NO: 24 - protein, 602 aa, Escherichia coli
  Escherichia coli colicin 1a, chain A
EIMAVDIYVNPPRVDVFHGTPPAWSSFGNKTIWGGNEWVDDSPTRSDIEKRDKEITAYKNTLSAQQKENENKRTEAGKRLSAAIAAREKDENTLKTLRAGNADAADITRQEFRLLQAELREYGFRTEIAGYDALRLHTESRMLFADADSLRISPREARSLIEQAEKRQKDAQNADKKAADMLAEYERRKGILDTRLSELEKNGGAALAVLDAQQARLLGQQTRNDRAISEARNKLSSVTESLNTARNALTRAEQQLTQQKNTPDGKTIVSPEKFPGRSSTNDSIVVSGDPRFAGTIKITTSAVIDNRANLNYLLSHSGLDYKRNILNDRNPVVTEDVEGDKKIYNAEVAEWDKLRQRLLDARNKITSAESAVNSARNNLSARTNEQKHANDALNALLKEKENIRNQLSGINQKIAEEKRKQDELKATKDAINFTTEFLKSVSEKYGAKAEQLAREMAGQAKGKKIRNVEEALKTYEKYRADINKKINAKDRAAIAAALESVKLSDISSNLNRFSRGLGYAGKFTSLADWITEFGKAVRTENWRPLFVKTETIIAGNAATALVALVFSILTGSALGIIGYGLLMAVTGALIDESLVEKANKFW

SEQ ID NO: 25 - protein, 602 aa, Artificial Sequence
  synthetic Escherichia coli colicin 1a, chain A variant polypeptide
EIMAVDIYVNPPRVDVFHGTPPAWSSFGNKTIWGGNEWVDDSPTRSDIEKRDKEITAYKNTLSAQQKENENKRTEAGKRLSAAIAAREKDENTLKTLRAGNADAADITRQEFRLLQAELREYGFRTEIAGYDALRLHTESRMLFADADSLRISPREARSLIEQAEKRQKDAQNADKKAADMLAEYERRKGILDTRLSELEKNGGAALAVLDAQQARLLGQQTRNDRAISEARNKLSSVTESLNTARNALTRAEQQLTQQKNTPDGKTIVSPEKFPGRSSTNDSIVVSGDPRFAGTIKITTSAVIDNRANLNYLLSHSGLDYKRNILNDRNPVVTEDVEGDKKIYNAEVAEWDKLRQRLLDARNKITSAESAVNSARNNLSARTNEQKHANDALNALLKEKENIRNQLSGINQKIAEEKRKQDELKATKDAINFTTEFLKSVSEKYGAKAEQLAREMAGQAKGKKIRNVEEALKTYEKYRADINKKINAKDRAARAAALESVKLSDISSNLNRFSRGLGYAGKFTSLADWITEFGKAVRTENWRPLFVKTETIIAGNAATALDALKFSKLTGSALGKKGYGDDMAVTGALIDESLVEKANKFW

SEQ ID NO: 26 - protein, 105 aa, Enterobacteria phage lambda
  Enterobacteria phage lambda holin
MPEKHDLLAAILAAKEQGIGAILAFAMAYLRGRYNGGAFTKTVIDATMCAIIAWFIRDLLDFAGLSSNLAYITSVFIGYIGTDSIGSLIKRFAAKKAGVEDGRNQ

SEQ ID NO: 27 - protein, 105 aa, Artificial Sequence
  synthetic Enterobacteria phage lambda holin variant polypeptide
MPEKHDKRAAILAAKEQGKGAKLAFAMAYLRGRYNGGAFTKTVIDATMCAKKAWFIRDLLDRAGLSSNLAYKTSVFKGYIGTDSKGSLIKRFAAKKAGVEDGRNQ

SEQ ID NO: 28 - protein, 6 aa, Artificial Sequence
  synthetic N-proximal His segment, 6xHis tag
HHHHHH

(19) United States
(12) Patent Application Publication
(10) Pub. No.: US 2013/0273585 A1
(43) Pub. Date: Oct. 17, 2013

(54) SOLUBLE CYTOPLASMIC EXPRESSION OF HETEROLOGOUS PROTEINS IN ESCHERICHIA COLI

(71) Applicant: GangaGen, Inc., Newark, CA (US)

(72) Inventors: C. B. Appaiah, Bangalore (IN); Sriram Padmanabhan, Bangalore (IN); R. Sanjeev Saravanan, Bangalore (IN)

(73) Assignee: GangaGen, Inc., Newark, CA (US)

(21) Appl. No.: 13/861,133

(22) Filed: Apr. 11, 2013

(30) Foreign Application Priority Data
Apr. 11, 2012 (IN) 1460/CHE/2012

(51) Int. Cl. C07K 1/14

(52) U.S. Cl. CPC C07K 1/145
USPC 435/18; 435/29; 530/300; 530/350

(57) ABSTRACT

Soluble variants of recombinant proteins produced in a prokaryotic host cell, where the high expression levels often cause the original proteins to aggregate into insoluble inclusion body aggregates. The variant polypeptides retain biological function while increasing protein solubility with comparable or higher recoverable levels of biologically active protein when expressed in a suitable expression host. Methods of identifying critical residues and substituting them are provided to produce the variants.

### CROSS-REFERENCES TO RELATED APPLICATIONS

The present disclosure incorporates by reference Indian Application No. 1460/CHE/2012 filed 11 Apr. 2012, the disclosure of which is incorporated herein by reference in its entirety.

Provided herein are soluble variants of recombinant proteins produced in a prokaryotic host cell, where the high expression levels often cause the original proteins to aggregate into insoluble aggregates. These variant polypeptides will retain biological function while increasing protein solubility with comparable or higher recoverable levels of protein when expressed in a suitable expression host.

### BACKGROUND OF THE INVENTION

Recombinant DNA technology has provided the means for large scale production of many proteins of medical or industrial importance. See, e.g., Alberts, et al. (2002) Molecular Biology of the Cell (4th ed.) Garland; and Lodish, et al. (1999) Molecular Cell Biology (4th ed.) Freeman. Large amounts of a protein can often be produced both simply and economically by recombinant DNA technology through expression of protein genes in prokaryotic production hosts. See, e.g., Sambrook and Russell (2001) Molecular Cloning: A Laboratory Manual (3 vol., 3d ed.), CSH Lab. Press; Scopes (1994) Protein Purification: Principles and Practice (3d ed.) Springer Verlag; Simpson, et al. (eds. 2009) Basic Methods in Protein Purification and Analysis: A Laboratory Manual CSHL Press, NY, ISBN 978-087969868-3; and Friedmann and Rossi (eds. 2007) Gene Transfer: Delivery and Expression of DNA and RNA, A Laboratory Manual CSHL Press, NY, ISBN 978-087969764-8. The efficient synthesis of heterologous proteins in the bacterium Escherichia coli has now become routine. However, when high expression levels are achieved, recombinant proteins are frequently expressed in E. coli as insoluble protein aggregates described as “inclusion bodies” (IB). A majority of recombinant proteins highly expressed in E. coli accumulate in inclusion bodies (i.e., protein aggregates). Most proteins in inclusion bodies are considered to be improperly folded or otherwise denatured, which generally means they are also substantially inactive enzymatically and/or may have compromised function. A substantial proportion of the protein from inclusion bodies is not recoverable into active form. The purification of the expressed proteins from inclusion bodies usually requires two main steps: extraction of inclusion bodies from the bacteria followed by the solubilization of the protein contained in the purified inclusion bodies. Typically, the proteins contained in the inclusion bodies, which are incorrectly folded, must be disaggregated and subsequently refolded efficiently into an active conformation. This is typically a cumbersome, difficult, and inefficient process. It would be much more desirable to highly express a soluble version of the recombinant protein.

A recombinantly expressed protein produced by a prokaryotic ribosome will often emerge in a sufficiently unusual microenvironment that it does not properly reach a soluble secondary or tertiary protein conformation. This often has fatal effects, especially if the intent of cloning is to produce an enzymatically active protein. For example, the internal microenvironment of a prokaryotic cell (pH, osmolarity, redox conditions, concentrations of cofactors and chaparones, etc.) will often differ significantly from that where the expression level is lower or occurs in the context of a more normal metabolic state. Various molecules or conditions allowing folding a protein at low expression levels may also be absent or limiting, and hydrophobic residues that normally would remain buried may be exposed and available for interaction with other exposed sites on other ectopic proteins. Protein processing systems or mechanisms may be overwhelmed at high expression levels or absent in particular bacteria production hosts. In addition, fine controls that may keep the concentration of a particular protein low or soluble at low expression levels may fail or be missing in a different prokaryotic producing cell, and overexpression can result in filling a cell with ectopic protein that, even if it were properly folded, would precipitate by saturating its environment.

One common strategy to avoid inclusion body formation is to fuse a protein segment of interest (i.e., the target protein segment) to a protein segment known to be expressed at substantial levels in soluble form in E. coli (i.e., the carrier protein segment). The soluble character of the carrier protein segment is hoped to counter issues causing the target protein segment to form inclusion bodies. LaVallie, et al. (1993) “A thioredoxin gene fusion expression system that circumvents inclusion body formation in the E. coli cytoplasm” Biotechnology 11:187-93, used thioredoxin as a carrier protein segment to express 11 human and murine cytokines, which are relatively short well behaved polypeptides. Of the 11 protein fusions, only 4 were expressed in soluble form as thioredoxin fusions at 37° C. Also, due to the small size of thioredoxin (11.7 kilodaltons) segment, fusions with larger protein segments may not be soluble; that is, thioredoxin may not be large enough to compensate for the insolubility of a large protein segment. Conversely, much of the protein produced by the expression system is the carrier sequence component of the fusion construct, which ultimately is not the desired function of the target protein segment and generally is removed and/or wasted. In either case, the production has produced a significant amount of extraneous polypeptide.

Thus, insolubility of target proteins in recombinant expression systems is a major problem in protein production or manufacturing. These affect the simplicity, ease of production, and economics of production and purification of the desired target function. The present disclosure addresses these and many other factors for many insoluble proteins.

### BRIEF SUMMARY OF THE INVENTION

The present disclosure is based, in part, upon the observation that many recombinant proteins produced in high level expression systems in E. coli hosts end up in insoluble inclusion bodies. Although high levels of protein are produced, often biological activity cannot be recovered because the protein cannot be renatured into a biologically active form in an easy way. Renaturation of proteins from inclusion bodies may be analogous to refolding denatured proteins, where recovery yields are typically very low. In particular, normal proteins will dynamically fold as they are synthesized from the ribosome beginning from the N terminus. As such, the active conformation of a protein assumes a kinetically optimal conformation, which may be different from the thermodynamically most stable form starting with a full length polypeptide. Thus, the N terminus folds in a microenvironment before the C terminal is synthesized.

Thus, there will often be factors which limit how quickly active conformation proteins can be produced. High level expression systems likely produce inclusion bodies when their polypeptide production rate exceeds the capacity of the limiting factor. Provided herein are methods to remove conformation folding limitations by changing the polypeptide sequences.

Provided herein are methods of identifying a variant protein of an insoluble first protein produced in a selected prokaryotic high expression system, the method comprising the steps of: (i) selecting a first protein which is insoluble when produced in the selected prokaryotic high expression system; (ii) identifying one or more residues in the protein which highly correlate with such insolubility; and (iii) substituting the amino acid residue with a less hydrophobic amino acid residue; thereby resulting in a variant protein which is recoverable in higher specific activity upon expression in the selected prokaryotic high expression system. In some embodiments, the residues which highly correlate with such insolubility: a) include highly hydrophobic residues in a segment of about 20 to 32 amino acids with a DAS score peak of at least about 2.3-2.5; or b) are substituted with one or more amino acids with a hydrophobicity score at least about 0.5 less than the substituted residue. In some embodiments, the insoluble first protein forms inclusion bodies, while the variant protein does not form inclusion bodies when analogously expressed in the same prokaryotic high expression system.

In some embodiments, the: a) residues which highly correlate with such insolubility include highly hydrophobic residues in a segment of about 19 to 31 amino acids with a transmembrane probability score of at least about 0.8 by TMHMM analysis; b) one or more is at least three; c) the first protein is biologically active, and the variant protein has a higher specific activity in a crude lysate upon expression in the selected prokaryotic high expression system; d) the first protein has 3 or fewer predicted transmembrane helices; e) the variant protein is expressed so that upon harvest and crude lysis, the variant protein is in active form in an amount at least about 3-10 fold higher than the first protein; f) less hydrophobic amino acid residue is an arginine, lysine, asparagine, glutamine, glutamic acid, or histidine; g) the first protein has a DAS score on the predicted transmembrane helix of more than about 2.3; h) the prokaryote high expression system comprises either batch or fed batch growth periods; i) the variant protein has substantially the same number of residues as the first protein; j) the first protein has a predicted transmembrane helix in the C terminus or middle portions; k) the amino acid residues include an isoleucine, valine, leucine, phenylalanine, cysteine, methionine, or alanine residue; 1) the prokaryote high expression system comprises a batch growth period; m) the prokaryotic high expression system comprises an inducible promoter; n) the amino acid residues include an isoleucine, valine, or leucine residue; o) the less hydrophobic amino acid residue is a proline, tyrosine, tryptophan, serine, or threonine; p) the first protein is less than about 300 amino acids; q) the less hydrophobic amino acid residue is a hydrophilic amino acid residue; r) the variant protein is an enzyme; s) the variant protein has at least 10× enzyme specific activity compared to the first protein in crude lysates when both are expressed in a similar high efficiency expression system; or t) the prokaryote is E. coli.

Further embodiments include the method wherein surface residue analysis is used to determine which residues which highly correlate with such insolubility are located at a location which interacts with the outer solvent, and a hydrophobic amino acid residue located at the location is substituted with a less hydrophobic residue. Among the more important embodiments here are where the: a) variant has substantially the same number of residues as the first protein; b) first protein does not have a fusion tag or fusion protein attached; or c) variant protein is an enzyme.

Further provided are variant polypeptides of a first polypeptide, wherein the first polypeptide is insoluble upon high expression conditions in a prokaryotic expression host, and the soluble variant: a) contains one or more substitutions of a less hydrophobic amino acid residue at one or more positions of the first polypeptide within a region of about 19-33 contiguous residues exhibiting a peak DAS score of at least about 2.3-2.5; and b) exhibits a higher biological specific activity per weight of such polypeptide than for the insoluble first polypeptide made in the prokaryotic expression host. In some embodiments, the: a) first polypeptide forms inclusion bodies in the high expression conditions; b) high expression conditions include a batch growth phase; c) one or more is at least three; d) the variant has a lower peak DAS score by at least about 0.3-0.5 than the first polypeptide; e) the variant has fewer than about 10% more residues than the first polypeptide; or f) the variant has biological specific activity during culture is at least about 3-7 fold greater than the first polypeptide.

Further provided are variant proteins of a first protein possessing a segment of about 20 to 35 amino acids which TMHMM analysis provides a transmembrane probability of at least about 0.7 and is insoluble upon high expression conditions in a prokaryotic expression host, the soluble variant protein: a) contains one or more substitutions of a less hydrophobic amino acid residue at one or more positions in the segment of the first protein; and b) exhibits a higher biological specific activity per weight of such protein made than for the insoluble first protein made in the prokaryotic expression host. In some embodiments, a) a corresponding segment of the variant protein to the segment of at least about 20 to 35 amino acids possessed by the first protein has a transmembrane probability score of less than about 0.6; b) the substitutions of a less hydrophobic amino acid residue include arginine, lysine, asparagines, aspartic acid, glutamine, glutamic acid, or histidine; or c) the variant protein can provide about 2-5 times more units of soluble biological activity per gram of cells than the first protein when both are produced in the high expression system conditions.

In certain circumstances, it will be desired to convert a soluble protein into a less soluble protein. As insoluble proteins are typically not enzymatically active, it may be desired to produce a protein toxic to its producing host cell in inactive form. In this embodiment, the protein may be converted from highly soluble to less soluble. Alternatively, a removable fusion construct can be added which causes the fusion construct to be insoluble, and the precipitated protein products can be isolated and converted into active form

### DETAILED DESCRIPTION OF THE INVENTION

The genomic and structural genomic communities have driven the development of high-throughput cloning and expression and purification technologies. The completion of genome sequencing of more than 100 organisms has opened up open-reading frames of numerous unknown functions. To understand the functions, such proteins are often expressed in the well studied host E. coli since it is easy to manipulate and is well characterized. See, e.g., Weickert, et al. (1996) “Optimization of heterologous protein production in E. coli” Curr. Opin. Biotechnol. 7:494-499. In certain cases, the studies use high throughput methodologies to produce hundreds of constructs and attempt to express them. See, e.g., Guan, et al. (2004) “High-Throughput Expression of C. elegans Proteins” Genome Res. 14:2102-2110. Most of these recombinant proteins are expressed in the cytoplasm, but many of them are difficult to express and purify due often to inhibitory effects on growth of host cells and/or the insolubility of the protein of interest. Overproduction of heterologous proteins in E. coli is especially challenging when one desires it to be soluble and functional and easy to purify. This is even more challenging when the protein of interest is composed of multiple subunits or is a membrane protein.

In most cases, inclusion body formation is a consequence of high expression rates, regardless of the system or protein used. It has been suggested that there is no correlation between the propensity of inclusion body formation with molecular weight, hydrophobicity, folding pathways, etc., except for proteins with disulphide linkages where the inclusion bodies are often formed due to scrambling of disulphides, whether intramolecularly or intermolecularly. See Lilie, et al. (1998) “Advances in refolding of proteins produced in E. coli” Curr. Opin. Biotechnol. 9:497-501. However, there is a common observation that hydrophobic proteins show aggregation upon over expression in bacterial cells. See, e.g., Shein and Noteborn (1988) “Formation of soluble recombinant proteins in Escherichia coli is favored by lower growth temperature” Bio/Technology 6:291-294.

Inclusion bodies do present problems, as described. In particular, the renaturation steps often use harsh reagents like guanidine hydrochloride, and urea for denaturation and refolding. The solubilization step also often requires several dilutions and many manipulations in the refolding, which typically makes for a complex and expensive process. The efficiency of successful refolding is always problematic, and loss of protein into improperly refolded product is typically a large fraction of the protein actually produced. Separation of improperly folded protein from properly folded active protein is generally also difficult. However, the inclusion bodies typically comprise at least 50% of the total cellular proteins, and generally contain the majority of the protein of interest. Thus, isolation of the inclusion bodies generally recovers most of the protein of interest.

Because of these problems with inclusion bodies, the economics of recombinant protein production has balanced the recovery yield of desired protein against simplicity of handling to achieve active protein. In most cases, the expression and purification conditions have been arrived at by trial and error. Typical strategies include changing the expression vector (see Cabrita, et al. (2006) “A family of E. coli expression vectors for lab scale and high through put soluble protein production” BMC Biotechnology 6:1-8); the expression temperature (to induce chaparones, both heat shock and cold shock forms help protein folding; most useful for where insolubility results from intermolecular interactions; see Weickert, et al. (1997) ‘Stabilization of apoglobin by low temperature increases yield of soluble recombinant hemoglobin in Escherichia coli” Appl. Environ. Microbiol. 63:4313-4320); targeting the protein to a different cellular compartment (which avoids association of the protein with the cell membrane) including targeting the protein into the periplasmic space away from cell membrane using appropriate signal sequences (see, e.g., Soares, et al. (2003) “Periplasmic expression of human growth hormone via plasmid vectors containing the lambda P1 promoter: use of HPLC for product quantification” Protein Engineering 16:1131-1138); selection of a host which favors production of correct pairing of disulfide linkages (for disulfide scrambling interactions; see Sørensen and Mortensen (2005) “Advanced genetic strategies for recombinant protein expression in Escherichia coli” J. Biotechnol. 115:113-28; using host strain which lacks thioredoxin reductase); and use of different types of promoters which may release proteins from ribosomes at a slower rate allowing kinetics of folding to occur differently (see, e.g., Qing, et al. (2004) “Cold-shock induced high-yield protein production in Escherichia coli” Nat. Biotechnol. 22:877-82; low temperature can improve protein expression; here cold shock promoters using the features of cspA gene to express proteins as soluble entities). Weak promoters such as constitutive promoters also often enhance solubility status of the expressed protein.

Another strategy is to link a target protein with fusion proteins or tags which can compensate for some of the physicochemical properties which lead to insolubility. Solubility enhancer fusion tags include the Maltose Binding Protein (MBP, see, e.g., di Guan, et al. (1988) “Vectors that facilitate the expression and purification of foreign peptides in Escherichia coli by fusion to maltose-binding protein” Gene 67:21-30); GST (see, e.g., .Smith and Johnson (1988) “Single-step purification of polypeptides expressed in Escherichia coli as fusions with glutathione S-transferase” Gene 67:31-40); thioredoxin (see, e.g., LaVallie, et al. (1993) “A thioredoxin gene fusion expression system that circumvents inclusion body formation in the E. coli cytoplasm” Biotechnology 11:187-93); NusA (see, e.g., Davis, et al. (1999) “New fusion protein systems designed to give soluble expression in Escherichia coli” Biotechnol. Bioeng. 65:382-88, and Harrison (1999) “Expression of soluble heterologous proteins via fusion with NusA protein” InNovations 11:4-7); intein; His tag (see, e.g., Hammarstrom, et al. (2001) “Rapid screening for improved solubility of small human proteins produced as fusion proteins in Escherichia coli” Protein Science 11:313-321; and Smith, et al. (1988) “Chelating peptide-immobilized metal ion affinity chromatography. A new concept in affinity chromatography for recombinant proteins” J. Biol. Chem. 263:7211-215); SUMO fusions; SerAsp (SD) repeats (see e.g., Banerjee and Padmanabhan “Novel fusion tag offering solubility to insoluble recombinant protein” WIPO Patent Application WO/2010/125588 2010); and a plethora of others. However, no universal method has been established for the efficient folding of aggregation prone recombinant proteins.

Recombinant protein production problems include: some proteins are extremely difficult to get soluble; wasted peptide production for larger fusion proteins; lack of success using shorter fusion tags; maintaining conformation of the target domains with fusion segment attached; molar ratio of fusion tag to protein produces lesser quantity of target protein; need often to remove the fusion segment from the target segment; need to use a cleavage enzyme to remove the fusion partner; need to demonstrate the absence of the same in the final end product, etc. Increasing solubility by limited mutagenesis can address these issues.

Another strategy for producing recombinant proteins in large quantities has been to use different host production systems. Examples include Bacillus species such as B. brevis or B. subtilis which secrete protein into the extracellular media. See, e.g., Yamagata, et al. (1989) “Use of Bacillus brevis for efficient synthesis and secretion of human epidermal growth factor” Proc. Natl. Acad. Sci. USA 86:3589-593; and Wang, et al. (1988) “Expression and secretion of human atrial natriuretic alpha-factor in Bacillus subtilis using the subtilisin signal peptide” Gene 69:39-47. Lactococcus lactis has been used for production of food-grade proteins. See, e.g., Morino, et al. (2008) “Lactococcus lactis, an efficient cell factory for recombinant protein production and secretion” J. Mol. Microbiol. Biotechnol. 14:48-58. Pseudomonas fluorescens has also been used. See, e.g., Retallack, et al. (2012) “Reliable protein production in a Pseudomonas fluorescens expression system” Protein Expr. Purif. 81:157-65. Rhodococcus erythropolis has been used (see Nakashima and Tamura (2004) “A novel system for expressing recombinant proteins over a wide temperature range from 4-35° C.” Biotechnol. and Bioeng. 86:136-148) as a Gram-positive host which can grow between 4-35 deg C., offering high temperature range culture operations. Eucaryotic cells like yeast cells, insect cells, mammalian cells may be used for achieving solubility, and may be necessary for glycosylated proteins and that require post-translational modifications. Mutant E. coli laboratory strains such as C41/C43 allow over expression of some globular and membrane proteins. See, e.g., Sorensen and Mortensen (2005) “Soluble expression of recombinant proteins in the cytoplasm of E. coli” Microbial Cell Factories 4:1-8. Folding and disulfide bond formation in the target protein may be enhanced by fusion to thioredoxin in strains that lack thioredoxin reductase (trxB). See, e.g., Sørensen and Mortensen (2005) “Advanced genetic strategies for recombinant protein expression in Escherichia coli” J. Biotechnol. 115:113-28. A heat-stable DNA binding protein has been reported to enhance recombinant protein expression by the binding of the same to the enhancer sequence and bending the DNA. See Richins, et al. (1997) “Elevated F is expression enhances recombinant protein production in Escherichia coli” Biotechnol. and Bioeng. 56:138-144. However, the coli production systems generally are most efficient high expression level producers when “efficiency” is measured by the quantitative amount of polypeptide produced. However, the “quality” of the resulting protein (when measured by biologically active protein yield) will often display lower yield than the engineered variants described here.

Cultivation Strategies:

Batch cultivation: All nutrients required for growth are supplied in the beginning culture. Cell densities are moderate and toxins accumulate over the culture period.

Fed batch: The concentration of energy sources is adjusted according to the rate of consumption. The formation of inclusion bodies can be followed in fed batch cultivations by monitoring changes in intrinsic light scattering by flow cytometry. This allows for real time optimization of growth conditions as soon as the inclusion bodies are detected, even at low levels, and inclusion body formation can potentially be avoided.

Folding of protein with co-factors: Addition of necessary cofactors may dramatically increase the yield of soluble proteins. Examples include addition of heme for expression of recombinant mutant of hemoglobin, as the cofactor seems to be limiting in the proper production of the protein. Similarly, a 50% increase in solubility was observed for glioshedobin when E. coli was induced in the presence of metal ions like magnesium. See Yang, et al. (2003) “High level expression of a snake venom enzyme, glioshedobin, in E. coli in presence of metal ions” Biotechnology Letters 25:607-610.

Low temperature induction: It has been suggested that reduction in the cultivation and induction temperature results in higher yields of soluble protein mainly due to decreased protein synthesis rate and in turn lesser protein aggregates. See Shein and Noteborn (1988) “Formation of soluble recombinant proteins in Escherichia coli is favored by lower growth temperature” Bio/Technology 6:291-294.

Addition of non-metabolizable carbon sources such as desoxy-glucose at the time of induction can result in reduced metabolic rate resulting in lesser protein expression, which may make the product remain soluble in cells.

### Molecular Modifications of Protein of Interest to Enhance Solubility:

Amino acid substitution is also one of the ways to enhance protein production in E. coli. This could be done by imparting changes in hydrophobicity or hydrophilicity of various positions of a polypeptide, e.g., by variation of the amino acids. The consequences of a given mutation would depend on the nature of the amino acid that is substituted and the environment in which it occurs. With deletions, the nature of the mutation is more complicated since the surrounding residues may all be affected as the protein backbone might need to shift to regain connectivity. Munishkin and Wool (Munishkin and Wool (1995) “Systematic deletion analysis of ricin A-chain function. Single amino acid deletions” J. Biol. Chem. 270:30581-587) were able to show that ricin is able to tolerate a wide array of deletions throughout the protein structure and still retain activity. Deletion of one or more amino acids was tolerated in all eight α-helices, all six β-strands, and all of the connecting loops. This work provides a dramatic illustration of the degree to which proteins may tolerate small deletions (typically two to five amino acids), often involving residues in the hydrophobic core, and yet still be able to assemble an active site and generate measurable catalytic activity.

Proteins are generally tolerant of certain amino acid substitutions. Studies of natural variants, as well as of proteins subjected to intensive mutagenesis, have revealed that many, possibly most, single amino acid substitutions are tolerated. This may be particularly so with conservative substitutions. Moreover, it appears that few, if any, residues in a protein cannot be replaced with at least one alternative amino acid. If combinations of substitutions are permitted, even the hydrophobic core of a protein can be packed in many different ways. Against this background of tolerance, certain positions in proteins stand out as particularly intolerant of substitutions. These critical residues are ones whose replacement with other residues frequently results in a loss of function.

In certain cases, amino acid insertions or deletions would achieve similar goals as substitutions. For example, where a number of clustered substitutions would be appropriate, an alternative would be to delete a hydrophobic stretch and substitute by insertion a less hydrophobic stretch of amino acids, which lengths might not be identical.

Examples where amino acid substitutions have caused loss of protein function:

Substitutions at positions in the hydrophobic strips of the T4 lysozyme led more frequently to loss of function than substitutions in the protein as a whole. See Rennell, et al. (1992) “Critical Functional Role of the COOH-terminal Ends of Longitudinal Hydrophobic Strips in a-Helices of T4 Lysozyme” J. Biol. Chem. 267:17748-17752).

Sickle cell anemia is an autosomal recessive genetic disorder. This is most commonly caused by the hemoglobin variant HbS where the hydrophobic amino acid valine takes the place of hydrophilic glutamic acid at the sixth amino acid position of the HBB polypeptide chain. This substitution creates a hydrophobic spot on the outside of the protein structure that sticks to the hydrophobic region of an adjacent hemoglobin molecule's beta chain. This clumping together (polymerization) of HbS molecules into rigid fibers causes the “sickling” of red blood cells. For the disease to be expressed, a person must inherit either two copies of Hb S variant or one copy of Hb S and one copy of another variant.

Alteration of a single leucine at position 344 to alanine (L344A) in the context of the amino-terminal fragment of a critical protein called VP16 of the Herpes simplex virus type 1 (HSV-1) abolished the interaction with virion host shutoff protein (vhs) that plays a role as a viral structural component, disabling host protein synthesis and triggering mRNA degradation following infection. Leu344 could be replaced with hydrophobic amino acids (Ile, Phe, Met, or Val) but not by Asn, Lys, or Pro, indicating that hydrophobicity is an important property of binding to vhs protein. See Knez, et al. (2003) “A Single Amino Acid Substitution in Herpes Simplex Virus Type 1 VP16 Inhibits Binding to the Virion Host Shutoff Protein and Is Incompatible with Virus Growth” J. Virol. 77:2892-2902.

Receptor activator of nuclear factor-κB ligand (RANKL), a trimeric tumor necrosis factor (TNF) superfamily member, is the central mediator of osteoclast formation and bone resorption. Functional mutations in RANKL lead to human autosomal recessive osteopetrosis (ARO), whereas RANKL over-expression has been implicated in the pathogenesis of bone degenerative diseases such as osteoporosis. See Douni, et al. (2012) “A RANKL G278R mutation causing osteopetrosis identifies a functional amino acid essential for trimer assembly in RANKL and TNF” Hum. Mol. Genet. 21:784-798.

The Mig1 repressor, a zinc finger protein that mediates glucose repression in Saccharomyces cerevisiae, has shown that two domains in Mig1p are required for repression: the N-terminal zinc finger region and a C-terminal effector domain, and it has been shown that four conserved residues within the effector domain, three leucines and one isoleucine, are particularly important for its function in vivo. See Östling, et al. (1998) “Four hydrophobic amino acid residues in the C terminal effector domain of the yeast MIG1P repressor are important for its in-vivo activity” Molec. Gen. Genetics 260:269-279.

Examples of recombinant proteins that do not get expressed in E. coli include but are not limited to: Saal; HADH4; Cytochrome b5e1; RIKEN1500015G18; transferring; apo A-V; cathepsin D; kallikrein 6; DNase I; pancreatic RNase; HMG-1; Kid I; Bax alpha; and glucokinase.

Examples of recombinant therapeutic proteins that are known to form inclusion bodies when expressed in E. coli: human granulocyte colony stimulating factor; human macrophage granulocyte colony stimulating factor; human interferon alpha 2a and interferon alpha 2b; human reteplase; human parathyroid hormone; interleukin-2; interleukin-11; growth hormone; human serum albumin; creatine kinase; urokinase; insulin; porcine phospholipase A2; epidermal growth factor; and platelet derived growth factor.

Examples of diagnostic proteins that do not get expressed in E. coli include but are not limited to: human enterokinase; GFP; FtsZ; FtsH; procathepsin D (Sachdev and Chirgwin (1998) “Solubility of proteins isolated from inclusion bodies is enhanced by fusion to maltose-binding protein or thioredoxin” Protein Expression and Purification 12:122-132); pepsinogen; actin (Frankel, et al. (1991) “The use of sarkosyl in generating soluble protein after bacterial expression” Proc. Natl. Acad. Sci. USA 88:1192-196); and banzonase. These are examples of proteins where conversion of sequence may lead to much simpler production and handling.

The effects of sequence variation will often be greater for shorter proteins. Because the density of thermodynamic effect is diluted for larger proteins, the methodology described herein may be more effective for smaller proteins. Thus, the protein may be more effected by substitutions when the protein is less than, e.g., about 600, 550, 500, or 450 amino acids, more likely for about 400, 350, 300, or 250 amino acids, and most likely to be applicable to proteins of less than about 200, 150, 125, or 100 amino acids. The method will also typically work best for fewer regions of hydrophobicity, and will apply well to proteins with fewer than 4 or 3 predicted transmembrane helices, and better to proteins with 2 or just 1 predicted transmembrane helix.

In addition, the location of predicted transmembrane helix in the protein may be relevant. The method may work particularly well for proteins where the predicted transmembrane helix is at the C terminus of the protein, or in the middle of the protein, or perhaps away from the N terminal region. In other cases, the method may be applicable to larger numbers of proteins where the predicted transmembrane helix is near or at the N terminus, which might include proteins where a signal sequence is not recognized in a translocation process across a membrane.

A “soluble” protein is one in solution in an appropriate buffer that does not form detectable precipitate. Generally the buffer is selected to be compatible with an assay for biological activity. One determination of whether protein is in solution is to test for insoluble aggregates or precipitates by centrifugation. Conversely, a protein is not soluble if at equilibrium the protein can be sedimented by centrifugation.

Inclusion bodies are aggregates of protein which form within producing cells upon high level expression conditions. The aggregates typically contain protein which is denatured or in an insoluble conformation.

A “Membrane Translocating Domain” is a segment of a protein which is hydrophobic, and often causes a recombinant protein containing it to be insoluble and precipitate upon recombinant expression into inclusion body aggregates. In certain constructs, a domain with hydrophobic properties is desired, e.g., to provide interaction with a membrane or to interact with a counterpart segment or domain on another protein.

“Prokaryote high expression system” is a combination of host cell, expression construct, and growth conditions under which the protein of interest is highly expressed. Typically, such systems are intended for recombinant expression of protein constructs, and the growth conditions often employ a high level promoter and conditions to increase protein expression. Such systems typically produce some 5, 10, 30, 70, 100× or more the expression level of the same protein construct in their native host cells. In most cases, the high expression system includes one of a heterologous and/or inducible promoter, production of a foreign protein in the prokaryote host cell, or production of a recombinant product.

A residue will “highly correlate with insolubility” if the solubility or insolubility of the protein product can be converted from one to the other by changing the nature of that residue, typically alone, or sometimes in combination with a small number of other residues.

The hydrophobicity rating of an amino acid is a number assigned to each amino acid, as indicated, or Kyte and Doolittle (1982); Biswas, et al. (2003) “Evaluation of methods for measuring amino acid hydrophobicities and interactions” J. Chromatog. A 1000:637-655; Eisenberg (1984). “Three-dimensional structure of membrane and surface proteins” Ann. Rev. Biochem. 53: 595-623; and Rose and Wolfenden (1993) Annu Rev. Biomol. Struct. 22:381-415.

“Recoverable”, in the context of protein activity, refers to whether the activity can be readily retrieved in by simple purification steps. In the context of physical protein, recovery may include physical protein which may be in conformation which is not biologically active. Soluble purification steps apply in the context of such proteins. Insoluble proteins will normally require that the protein be refolded, which typically results in physical protein in a combination of soluble (and active) conformation form, soluble (and inactive) form, and insoluble inactive conformation forms.

“Higher specific activity” is a comparison of the specific activity of two protein preparations at useful protein concentrations, e.g., around 100 μg/ml. Typically, it can be achieved either by increasing an enzymatic activity attributable to a fixed amount of protein, or by removal of inactive protein which decreases the total amount of relevant physical protein.

“Upon expression”, or “during culture” refer to amounts active protein produced in the culture phase of expression. In comparing soluble protein produced to insoluble protein, the product of interest is recoverable activity. Thus, with a soluble protein, the recoverably activity may be greater even if the total amount of physical protein produced is less, especially where larger amounts of protein produced in inclusion bodies do not yield polypeptide which will exhibit the desired functional activity.

DAS scores are plotted for segments across a polypeptide. The “peak score” is the local maximum score which applies to adjacent segments in a region of the polypeptide.

“Analogously expressed” refers to comparing expression of different variants under the same expression conditions. Thus, in batch mode, the same conditions of culture are being compared. In fed batch mode, the same conditions and parameters for culture are applied for both constructs for comparison of yield or recovery, generally of functionally active protein.

“Highly correlate” is a relative term, in that the correlation is higher than selected alternatives.

“Highly hydrophobic residue” is a relative term. Hydrophobicity can be quantitatively ranked and assigned various measures by relevant software applications. See above and Table 1. Hydrophobicity is often assigned measures for each amino acid, as described below, e.g., between 4.5 to −4.5 in commonly used measures.

TABLE 1
Relative hydrophobicity measures

| Kyte and Doolittle | Rose, et al. | Wolfenden, et al. | Janin (1979) |
|---|---|---|---|
| Ile | Cys | Gly, Leu, Ile | Cys |
| Val | | Val, Ala | Ile |
| | Phe, Ile | | Val |
| Leu | Val | Phe | Leu, Phe |
| | Leu, Met, Trp | Cys | Met |
| Phe | | Met | Ala, Gly, Trp |
| Cys | | | |
| Met, Ala | His | Thr, Ser | |
| | Tyr | Trp, Tyr | His, Ser |
| Gly | Ala | | Thr |
| Thr, Ser | Gly | | Pro |
| Trp, Tyr | Thr | | Tyr |
| Pro | | | Asn |
| | | Asp, Lys, Gln | Asp |
| His | Ser | Glu, His | Gln, Glu |
| Asn, Gln | Pro, Arg | Asp | |
| Asp, Glu | Asn | | |
| Lys | Gln, Asp, Glu | | |
| | | | Arg |
| Arg | Lys | Arg | Lys |

Kyte and Doolittle (1982) J. Mol. Biol. 157: 105-132.
Rose, et al. (1985) Science 229: 834-838.
Wolfenden, et al. (1981) Biochemistry 20: 849-855.
Janin (1979) Nature 277: 491-492.

“At least 3” in the context of integral measures means 4, 5, 6, etc. Analogously for another integer “n”, at least n means integral numbers n or greater than n. Thus, a protein which comprises “at least 2” transmembrane segments will have 2, 3, 4, or more hydrophobic segments.

A segment of a polypeptide is a stretch of a number of residues, typically having a relevant length. In the context of a transmembrane helix, various software programs assign common assumptions as to length based on common occurrences. Most transmembrane segments are at least about 17-23 residues, but may be shorter or longer by a few residues. While a transmembrane helix may be structural, for solubility purposes the interaction of the segment with other protein segments may not be as limited to span a bilayer. Thus, longer or shorter segment lengths may be important in the context of protein solubility. Thus, segment lengths as short as about 12, 13, 14, etc., may be important in identifying hydrophobic segments, they may also be longer and may extend to about 23, 25, 27, 29, 31, 33, or 35 or more residues.

“Upon harvest” relates to crude recovery of proteins evaluated at the first steps after limited purification of soluble protein, and after isolation of inclusion bodies and first steps to solubilize. Typically, this is evaluated before inclusion body material is refolded. Evaluation requires that protein is recovered at a reasonable and useful protein concentration, e.g., at least 100 μg/ml, and preferably 300 or more.

Crude lysates refer to culture preparations where cells are harvested, sometimes washed to remove media, and the cells disrupted, thereby releasing the cell contents. The resulting crude lysates typically are prepared in buffer to maintain neutral pH and preserve desirable enzyme activity, but with minimal further purification of cell contents. Inclusion bodies present within the intact cells typically remain in inclusion bodies.

“Substantially same number of residues” means that protein lengths are similar, e.g., there are not dramatic differences in length. Thus, where a fusion protein or fusion tag is attached, the proteins with and without the fusion will not be substantially the same number of residues.

An “enzyme” possesses a biologically relevant and useful activity exhibited by the polypeptide. Occasionally a cofactor or such might be necessary to be attached, and the efficiency of such modification applies to different variants being compared.

An N terminal transmembrane segment is a transmembrane segment, typically indicated as a transmembrane helix, which may be predicted or physically determined, which is at the N proximal portion of the sequence of the subject protein. Analogously, a C terminal transmembrane segment would be at the C proximal portion of the sequence of the subject protein. In this context, the middle of the protein would be between the N proximal and C proximal sections. It should be noted that in certain circumstances, the location of a transmembrane helix, whether amino or carboxy proximal, may be important in either the kinetics or thermodynamics of polypeptide folding. Protein folding from the ribosome is a dynamic temporal process, which progresses as the polypeptide is synthesized.

“Surface residue analysis” is a methodology used to determine what regions (location of peptide, amino acid residues) of a properly folded polypeptide sequence are exposed to the surface of the structure and interact with solvent in which the protein is dissolved.

“Higher biological specific activity per weight of polypeptide made” refers to a comparison of total “biological activity per weight” of physical protein present. In many cases, physical protein may be present in a conformation where no enzymatic activity is exhibited, and the specific activity is diluted from the larger denominator from the inactive protein. Comparison of specific activities will typically detect differences of 10%, 20%, 30%, 50% or more, though greater differences, e.g., 2×, 3×, 5×, 7×, 10× or more in comparison to a native or unmodified protein will be effected by changes in the solubility of variants.

The “TMHMM transmembrane probability” (TMHMM) output provides a quantitative number of transmembrane probability, which typically complements the score corresponding to probability of the segment being found inside the cell. Similar evaluations with other software provide prediction of whether particular segments of polypeptide sequence are likely to interact with lipids or span typical membranes. In other cases, the prediction of transmembrane segments can also indicate likelihood of sufficient hydrophobicity to interact with other hydrophobic segments, whether intramolecularly, intermolecularly, or with another hydrophobic region, e.g., a membrane.

Methods to Determine Soluble Versus Insoluble Proteins:

One-milliliter samples are withdrawn into Eppendorf tubes at appropriate times after induction. These 1 ml samples are centrifuged in an Eppendorf centrifuge at 4 deg C. for 3 min, and the supernatants are removed. The pellets are stored at −80 deg C. until they are assayed. Soluble and insoluble contents are determined, see Weickert and Curry (1997) “Turnover of recombinant human hemoglobin in Escherichia coli occurs rapidly for insoluble and slowly for soluble globin” Arch. Biochem. Biophys. 348:337-46. In brief, the cell density in fermentation samples is determined directly or calculated from the measured cell density. Cells are lysed by lysozyme addition and incubation on ice, and the DNA is digested with DNase. The soluble and insoluble fractions are separated by centrifuging the lysate for 15 min in a microcentrifuge at top speed. The supernatant (soluble fraction) is transferred to another microcentrifuge tube, except that after sodium dodecyl sulfate-polyacrylamide gel electrophoresis, the rHb is detected by either silver staining or Western blotting. The gels are silver stained by using the reagents and protocol recommended by Daiichi Pure Chemicals Co., Ltd. (Tokyo, Japan).

Inclusion bodies are dense particles of aggregated proteins. Because of their refractile property, they can be visualized by light microscopy or assayed by other methods. See, e.g., Grimm, et al. (2004) “A rapid method for analyzing recombinant protein inclusion bodies by mass spectrometry” Anal. Biochem. 330:140-144. Structural analysis of the inclusion bodies indicate that the aggregated proteins have a certain amount of secondary structure as seen for in-vitro aggregated proteins. Oberg, et al. (1994) “Native like secondary structure in interleukin-1 beta inclusion bodies by attenuated total reflectance FTIR” Biochemistry 33:2628-2634.

Inclusion bodies can be easily pelleted by centrifugation due to their dense nature (1.3 mg/ml). See, e.g., Mukhopadhyay (1997) “Inclusion bodies and purification of proteins in biologically active forms” Adv. Biochem. Eng. Biotechnol. 56:61-109. Distinguishing inclusion bodies or insoluble protein aggregates from soluble proteins may be achieved by lysis of the induced bacterial cells by sonication followed by centrifugation at 1300 rpm (about 15K×g) for about 15 minutes. Inclusion bodies will sediment, while soluble proteins remain in solution. Generally, when a protein is in inclusion bodies in a host cell, the induced cell pellet after lysis by sonication does not decrease OD600 of the cell suspension much more than 2-3 fold, the inclusion bodies remaining in aggregated state. If protein is soluble, the culture OD600 during sonication drops by at least 10 folds. Similar differentiation methods are applicable based upon optical absorption of the inclusion bodies compared to protein solutions.

Alternatively, commercial extraction methodologies can separate insoluble forms of protein from soluble proteins. See, e.g., B-PER® and B-PER® II reagents (Pierce, USA), Zhou, et al. (2012) “Enhancing solubility of deoxyxylulose phosphate pathway enzymes for microbial isoprenoid production” Microbial Cell Factories, 11:148, and ReadyPrep protein extraction kit (BioRad, USA), Zhu, et al. (2012) “Characterization of a female-specific protein from the wild silkworm Actias selene” Bulletin of Insectology 65:107-112).

Aggregation and protein precipitation, which cause the solution to become cloudy because of insoluble aggregates, is important to avoid because once begun, the insoluble aggregates progressively grow and cause protein losses during storage and processing. Reducing irreversible protein adsorption translates to greater recovery in purification steps and improved efficiency of downstream processing and overall production. Moreover, the higher recovery of physical protein typically reflects more active conformation protein and lower amounts of inactive conformation protein. Copurifying inactive protein adversely affects the economics of production, and may affect dosage and other pharmacological parameters.

The hydrophobic nature of amino acids such as alanine, valine, leucine, isoleucine, proline, phenylalanine, tryptophan, cysteine, and methionine are recognized. While glycine does not have a side chain, it is often found on the surface of the protein tertiary structure in loop regions and provides additional flexibility to these regions and proline provides rigidity to the protein structure, by imposing certain torsion angles on the segment of the polypeptide chain where it is located. Thus, modifying the polypeptide sequence to minimize the insolubility can be applied by substituting highly hydrophobic amino acids at the protein surface to more polar or neutral amino acids.

The extent of protein adsorption can correlate with hydrophobicity of the protein. See Tilton, Robertson, and Gast (1991) “Manipulation of hydrophobic interactions in protein adsorption” Langmuir 7:2710-2718.

Hydrophilicity also has been reported to play a role in protein solubility. Instead of targeting only hydrophobic residues, another alternate would be to target the hydrophilic residues where the exercise would be to substitute the least or lesser hydrophilic residues with higher hydrophilic residues. See, e.g., Yan, et al. (2006) “A mutated human tumor necrosis factor-alpha improves the therapeutic index in vitro and in vivo” Cytotherapy 8:415-23. It was reported that hydrophilic residues were targeted to modify the proline, serine, and alanine of a Tumor Necrosis Factor (TNF) is replaced by residues with higher hydropathy index, like RKR.

As observed in Example 2, the hydrophobicity of the MTD may be such that the resulting protein product is insoluble within the cell upon synthesis. However, in certain cases, constructs can be generated which exhibit a combination of features which would otherwise be considered impossible. In particular, there are constructs which can be sufficiently hydrophilic to remain soluble within the producing cell host, while retaining the MTD function to traverse the bacterial outer cell wall, but lacking the MTD function to traverse the bacterial cell membrane. This may be achieved because the bacterial cell membrane properties (and structure) are sufficiently different from the bacterial outer membrane.

In this context, one selects constructs which combine the three properties: (1) produced in the appropriate bacterial cell host, typically Gram-negative E. coli, in substantially soluble form intracellularly; (2) retains function so the MTD effects the product to traverse the bacterial outer cell wall to access the periplasmic space where the substrate peptidoglycan is accessible to the catalytic domain; and (3) the MTD does not allow the soluble product to traverse the producing cell bacterial cell membrane to allow the catalytic domain to hydrolyze the peptidoglycan of the producing host cell. Appropriate controls will be incorporated to ensure that cell survival, expression, and catalytic activity can be quantitated.

As the aqueous solubility of a protein depends mostly on its hydrophilicity (or conversely, its lack of regions of great hydrophobicity), a protein which possesses regions of concentrated hydrophobicity may often be made more soluble by disrupting such stretches. As the MTD segments will typically be among the most hydrophobic segments of a construct, those regions will typically be of most interest.

With certain insoluble constructs from these chimeras, the MTD segment is a short transmembrane segment. The different hydrophobicity analyses are reasonably accurate in identifying relatively short transmembrane segments, which typically span about 20 amino acid residues. These are the target residues to modify to affect solubility of many proteins. Disrupting the membrane interaction of protein products can help avoid association with the inner cytoplamic membrane of the producing host cell. Otherwise decreasing the overall hydrophobicity of these regions will often change the overall protein solubility.

Amino acids with electrically charged side chains: Arg, H is, Lys: positive charge: hydropathy score being −4.5, −3.2, −3.9; Glu, Asp: negative charge being −3.5, −3.5. Amino acids with polar but uncharged side chains: Ser, Thr, Asn, Gln: hydropathy score being −0.8, −0.7, −3.5, −3.2. Amino acids with non-polar (hydrophobic side chains): Ala, Ile, Leu, Met, Phe, Trp, Tyr, Val: hydropathy score being 1.8, 4.5, 3.8, 1.9, 2.8, −0.9, −1.3, 4.2. For valine replacement, the substitutions would preferably be tyrosine or tryptophan to maintain the class of amino acid; if hydrophobicity is to be minimized replacement is preferably with arginine, histidine, or lysine. For isoleucine replacement, the substitutions would preferably be tyrosine or tryptophan to maintain the class of amino acid; if hydrophobicity is to be minimized replacement is preferably with arginine, histidine, or lysine. For leucine replacement, the substitutions would preferably be tyrosine or tryptophan to maintain the class of amino acid; if hydrophobicity is to be minimized replacement is preferably with arginine, histidine, or lysine.

Proline residues in hydrophobic stretches strongly disfavor the translocation arrest of transmembrane domains (TMDs) and favor the transfer of preproteins to the matrix. Meier, et al. (2005), “Proline residues of transmembrane domains determine the sorting of inner membrane proteins in mitochondria” J Cell Biology 170:881-888. Also, proline residues can break a transmembrane helix, but only when inserted near the end, and only when the helix is sufficiently long. Nilsson, et al. (1998) “Proline-induced Disruption of a Transmembrane alpha Helix in its Natural Environment”. J Mol Biol, 284, 1165-1175. Hence substitutions with proline should be avoided in such modifications.

Using DAS TMD analysis (see, e.g., Cserzo, et al. (1997) “Prediction of transmembrane α-helices in prokaryotic membrane proteins: the dense alignment surface method” Protein Engineering 10:673-676), TMHMM analysis (see, e.g., Krogh, et al. (2001) “Predicting transmembrane protein topology with a hidden Markov model: application to complete genomes” J. Mol. Biol. 305:567-580), general hydrophobicity (see, e.g., Kyte and Doolittle (1982) “A simple method for displaying the hydropathic character of a protein” J. Mol. Biol. 157:105-132), or the Grand Average of Hydropathy Score (GRAVY; see Gasteiger, et al (2005) “Protein Identification and Analysis Tools on the ExPASy Server” in Walker (ed. 2005) The Proteomics Protocols Handbook, Humana Press, pp. 571-607), regions of high hydrophobicity are identified. These are targeted to decrease extreme hydrophobicity, which often lead to protein interactions between polypeptides resulting in protein aggregation and precipitation of insoluble aggregates. Alternatively, stretches of hydrophobic residues may interact with membranes and lipid containing structures, preventing a polypeptide chain from achieving a normal soluble conformation.

DAS Prediction Server

The Dense Alignnment Surface (DAS) prediction server is meant for predicting transmembrane helices in membrane proteins. The program uses the condition that membrane proteins are composed of stretches of 15-30 predominantly hydrophobic residues separated by polar connecting loops. This means that the transmembrane region will detect a fragment that is predominantly composed of hydrophobic amino acids, flanked by residues that are hydrophilic or polar residues.

DAS is based on low-stringency dot-plots of the query sequence against a collection of non-homologous membrane proteins using a previously derived, special scoring matrix. Since integral membrane proteins are composed of more hydrophobic residues than water soluble globular proteins, they can be discriminated according to their composition. The principal difference between the DAS method and the hydrophobicity profile based programs is that DAS describes the hydrophobic segments at three levels. This complex approach of hydrophobicity is the key behind the sensitivity of the DAS method.

There are two cutoffs indicated on the plots: a “strict” one at 2.2 DAS score, and a “loose” one at 1.7. The hit at 2.2 is informative in terms of the number of matching segments, while a hit at 1.7 gives the actual location of the transmembrane segment.

TMHMM (TransMembrane Prediction by Hidden Markov Model)

TMHMM is a software analysis based on a hidden Markov model (see, e.g., the websites at cbs.dtu.dk/services/TMHMM/ and bioperl.org/wiki/TMHMM, and Krogh, et al. (2001) J. Mol. Biol. 305:567-80). It predicts transmembrane helices and discriminates between soluble and membrane proteins with a high degree of accuracy. Methods for prediction of transmembrane helices using hydrophobicity analysis alone are not reliable always. This method implicitly combines the hydrophobic signal to detect transmembrane (TM) segments and the charge bias, an abundance of positively charged residues in the part of the sequence on the cytoplasmic side of the membrane protein into one integrated algorithm. Also Helical membrane proteins follow a “grammar” in which cytoplasmic and non-cytoplasmic loops have to alternate. TMHMM can incorporate hydrophobicity, charge bias, helix lengths, and grammatical constraints into one model for prediction. This program allows one to predict the location of transmembrane alpha helices and the location of intervening loop regions together with prediction of which loops between the helices will be on the inside or outside of the cell or organelle. This program does not detect beta sheet transmembrane domains. It takes about 20 amino acids to span a lipid bilayer in an alpha helix. Programs can detect these transmembrane domains by looking for the presence of an alpha helix at least about 20 amino acids long which contains hydrophobic amino acids. It correctly predicts 97-98% of the transmembrane helices while Dense Alignment Surface method (DAS) to predict transmembrane segments in any integral membrane protein. DAS has two levels of stringency which is more comprehensive than TMHMM.

Kyte-Doolittle

A Kyte-Doolittle hydropathy plot gives information about the possible structure of a protein. A hydropathy plot can indicate potential transmembrane or surface regions in proteins (see, e.g., the websites at gcat.davidson.edu/rakarnik/KD.html and vivo.colostate.edu/molkit/hydrophathy/index.html). This does not predict secondary structure, so it will detect both alpha helix and beta sheet transmembrane domains. Numbers greater than 0 indicate greater hydrophobicity, while numbers less than 0 indicate greater hydrophilic measure of amino acids.

First, each amino acid is given a hydrophobicity score between 4.6 and −4.6. A score of 4.6 is the most hydrophobic and a score of −4.6 is the most hydrophilic. After a window size is set, it is the number of amino acids whose hydrophobicity scores will be averaged and assigned to the first amino acid in the window. The default window size is 9 amino acids. The computer program starts with the first window of amino acids and calculates the average of all the hydrophobicity scores in that window. Then the computer program moves down one amino acid and calculates the average of all the hydrophobicity scores in the second window. This pattern continues to the end of the protein, computing the average score for each window and assigning it to the first amino acid in the window. The averages are then plotted on a graph. The y axis represents the hydrophobicity scores and the x axis represents the window number. These values should be used as a rule of thumb and deviations from the rule may occur.

The Kyte-Doolittle scale is widely used for detecting hydrophobic regions in proteins. Regions with a positive value are hydrophobic, negative values are more hydrophilic. This scale can be used for identifying both surface-exposed regions as well as transmembrane regions, depending on the used window size. Short window sizes of 5-7 generally work well for predicting putative surface-exposed regions. Large window sizes of 19-21 are well suited for finding transmembrane domains if the values calculated are above about 1.6. These values should be used as a rule of thumb and deviations from the rule may occur.

GRAVY

The GRAVY score is the average hydropathy score for all the amino acids in the protein. According to Kyte and Doolittle (1982), integral membrane proteins typically have higher GRAVY scores than do globular proteins. Though this score is another helpful piece of information, it cannot reliably predict the structure without the help of hydropathy plots. This index is the general average hydropathicity (GRAVY) score for the hypothetical translated gene product. It is calculated as the arithmetic mean of the sum of the hydropathic indices of each amino acid.

Software to calculate GRAVY score is available free online on expasy Protparam (see the webite at web.expasy.ort/protparam/). The input is the amino acid primary sequence in single letter format. Since the score is an average value the parameter to be selected is the window size to adjust the number of amino acids that are averaged to obtain an individual hydropathy score.

Malen, et al. (Malen, et al. (2010) BMC Microbiology 10:132) reported that a substantial proportion of the detected proteins that had a negative GRAVY score were soluble proteins. However, they also suggest that at least some of them might be functionally membrane-associated through formation of protein complexes with membrane-anchored proteins. Also, several hydrophilic proteins are retained in the lipophilic membrane fraction due to interaction with hydrophobic proteins and the correlation between GRAVY score and solubility is not always correct. See, e.g., Althage, et al. (2004) “Cross-linking of transmembrane helices in proton-translocating nicotinamide nucleotide transhydrogenase from Escherichia coli: implications for the structure and function of the membrane domain” Biochim. Biophys. Acta 1659:73-82.; Guenebaut, et al. (1997) “Three-dimensional structure of NADH-dehydrogenase from Neurospora crassa by electron microscopy and conical tilt reconstruction” J. Mol. Biol. 265:409-418; and Guenebaut, et al. (1998) “Consistent structure between bacterial and mitochondrial NADH:ubiquinone oxidoreductase (complex I)” J. Mol. Biol. 276:105-112. There was no relationship between successful expression and protein pI, grand average of hydropathicity (GRAVY), or sub-cellular location. Dyson, et al. (2004) “Production of soluble mammalian proteins in Escherichia coli: identification of protein features that correlate with successful expression” BMC Biotechnology 4:32. According to Dyson (2004), GRAVY simply calculates overall hydrophobicity of the linear polypeptide sequence with increasing positive score indicating greater hydrophobicity, but no account is taken of the order of residues, the way the protein folds in three dimensions, or the percentage of residues buried in the hydrophobic core of the protein. In a recent study Luan, et al. (Luan, et al. (2004) “High-Throughput Expression of C. elegans Proteins” Genome Res. 14:2102-2110) tested the soluble expression of 10,167 full-length C. elegans ORFs and found that protein hydrophobicity was an important factor for an ORF to yield a soluble expression product. This different result may be attributable to the fact that the C. elegans study included a greater proportion of membrane proteins. Therefore the lack of correlation between GRAVY score and soluble expression we observed may be true for non-membrane proteins or for proteins where the transmembrane domain has been deleted.

| | GRAVY |
|---|---|
| | SCORE |
| BPI TMD SEQ ID NO: 2 | |
| Wild Type BPI TMD Sequence: A228 to R251 | 1.658 |
| Variants (orig AA; position number; replacement AA): | |
| V232E; V234D; I236K | 0.667 |
| V232K; V234K; I236R; V240K; V244K; V248K; V249K; | −1.104 |
| V250R | |
| V232K; V234K; I236R; V240K; V244K; V248K; V250R | −0.161 |
| L230R; I236R; V240K; V250R | 0.237 |
| P134 TMD SEQ ID NO: 5 | |
| Wild Type Sequence P134 TMD E242 to L264 | 1.774 |
| Variants (orig AA; position number; replacement AA): | |
| V250R; L251P | 1.161 |
| I243R; V250R; V256R; I261R | 0.235 |
| I243K; A248K; A249K; V250R; L251R; V256K; I261D | −0.526 |
| L246R; I261N; L264K | 0.730 |

In these types of analyses, typically amino acid residues are assigned hydrophobicity measures according to their physicochemical properties. These programs generally assign values such as: residue type, kd Hydrophobidity: Ile, 4.5; Val, 4.2; Leu, 3.8; Phe, 2.8; Cys, 2.5; Met, 1.9; Ala, 1.8; Gly, −0.4; Thr, −0.7; Ser, −0.8; Trp, −0.9; Tyr, −1.3; Pro, −1.6; His, −3.2; Glu, −3.5; Gln, −3.5; Asp, −3.5; Asn, −3.5; Lys, −3.9; Arg, −4.5.

The residue substitution strategy is to decrease peak regional hydrophobicity, e.g., where the DAS peak measure is above about 3.5 for the P266. The segment is modified to decrease the local DAS profile score. Thus, for various proteins, one targets the substantial peaks, which may peak at above about 3.1, or 2.9, 2.7, 2.5, or 2.2. Preferably the modifications can lower local peak values to less than about 2.2, 2.1, 2.0, 1.8 or perhaps even as low as about 1.5. Thus, target decreases in DAS profile score will preferably be at least about 0.2 units, more preferably about 0.3 or 0.4 units, or most preferably at least 0.5 units.

Similar corresponding changes in the transmembrane probability scores by the TMHMM would be desired. In the local scoring, the transmembrane probability would preferably be decreased from about 0.5, 0.6, 0.7, 0.8, or even 0.9 down to lower values. Conversely, the intracellular probability numbers would be increased. Target numbers may be down in the 0.6 or lower ranges, with drops of about 0.2, 0.3, or preferably 0.4 or 0.5.

Similar decreases in hydrophobicity are targeted by Kyte-Doolittle or GRAVY local measures.

Because the DAS and TMD analyses evaluate clusters of contiguous amino acids, local chain lengths may be varied. Where high measures of clustered hydrophobicity are found, the most hydrophobic residues are identified, typically ile, val, leu, and phe. Among these, various hydrophobic amino acids are selected individually or in combinations, for replacement or substitution by a less hydrophobic residue, either a more neutral or polar amino acid. A reasonable number of variants are constructed for screening for the combination of properties as described above.

Methods for Identifying Soluble Variants

The method for identifying soluble variants of insoluble proteins generally includes a series of steps. These generally include steps directed to identifying proteins for which the method may be applicable or relevant, identifying target segments of the protein to incorporate variations likely to affect aqueous solubility, generating such variant(s), and confirming solubility of protein products. In certain circumstances, the introduced changes may be evaluated to determine changes or combinations which may confer solubility while minimizing the number of changes.

The subject method is applicable to proteins which are insoluble, particularly where insolubility results in part from segments of polypeptide which are hydrophobic. The method is based, in part, upon the observation that segments of hydrophobicity correlate with insolubility of the product. Observations support that many proteins which form inclusion bodies do so as a result of interactions of hydrophobic stretches of polypeptide with other hydrophobic environments, e.g., similar hydrophobic segments of proteins accessible in the cytoplasmic environment or with lipid membranes. Examples include integral and surface membrane proteins for expression in prokaryote expression systems, e.g., bacterial and mammalian membrane proteins. Such membrane proteins often are attached directly to cell membrane, which may be receptors for signal transduction and other functions. Some integral membrane proteins include transporters, linkers, channels, enzymes, structural membrane-anchoring domains, proteins involved in accumulation and transduction of energy, proteins as phage receptors and proteins responsible for cell adhesion. Annotations of such proteins suggest the method may be applicable. A classification of transporters can be found in Transporter Classification database. Peripheral membrane proteins are temporarily attached either to the lipid bilayer or to integral proteins by a combination of hydrophobic, electrostatic, and other non-covalent interactions. See, e.g., Saier, et al. (2009) Nucleic Acids Res. 37 (database issue): D274-8. Other criteria may include proteins with relatively high hydrophobic residues in a clustered patch or distributed over a relatively short stretch, e.g., from 6-30, preferably 10-28, or more preferably 17-24 contiguous residues.

Another useful indicator is a protein with lesser amounts of charged amino acids, such as lysine and arginine. These amino acids are less frequent in integral membrane proteins and nearly absent in transmembrane helices. Since these amino acids are also cleavage targets for the common proteases such as trypsin or other host proteases, such amino acids are not present naturally.

Once a protein is selected for conversion from insoluble in an aqueous solvent into soluble, locations for where to introduce variations need to be identified. If the protein has a desired function, residues are selected which are unlikely to affect such.

Regions of highest hydrophobicity are identified, particularly ones which significantly affect aqueous solubility. Various software analyses accurately can predict the solubility of proteins based upon sequence. Among the more accurate programs are the TMHMM and the DAS, when the outputs and sequences are properly evaluated. The TMHMM software provides relatively accurate predictions of segments of protein which would form a transmembrane helix. The prediction correlates highly with sufficiently long segments of hydrophobicity that the proteins will often be insoluble when produced in a prokaryote high expression system. In a normal protein, typically hydrophobic amino acid residues are likely to be found clustered in the interior of a globular protein, while hydrophilic amino acid residues are exposed to interact with the aqueous cytoplasm. However, if hydrophobic residues are at the globular surface, those residues are likely to associate either with a membrane or similar hydrophobic segment of a protein, which may be intra or intermolecular. Such will often lead to aggregation of the polypeptides, leading to insoluble aggregates.

The various software programs use both empirical methods and thermodynamic features of the residues to predict when the proteins actually exhibit topological features in relation to membranes. Alternatively, different measures of hydrophobicity may be used with corresponding thresholds. For example, one measure assigns numbers between 4.5 and −4.5 (see above), while other “normalized” measures may be applied.

In one such alternative, the hydrophobicity index or values for various amino acids given are normalized so that the most hydrophobic residue is given a value of 100 relative to glycine, which is considered neutral (0 value). The scales were extrapolated to residues which are more hydrophilic than glycine. At pH 7.0, the most hydrophobic amino acids are leu (100), Ile (99), Phe (97), try (97), val (76), met (74), while the hydrophobic amino acids are Cys (63), Tyr (49), ala (41). The neutral amino acids are thr (13), His (8), Gly (O), ser (−5), gln (−10), and the hydrophilic amino acids are Arg (−14), Lys (−23), Asn (−28), Glu (−31), pro (−46), and asp (−55). See, e.g., sigmaaldrich.com.

Such measures of hydrophobicity are used to select residues that should be targeted for substitutions, or occasionally deletions or insertions. See, e.g., Monera, et al. (1995) J. Protein Sci. 1:319-329. The substitutions could be done in such a way that an amino acid with a positive hydrophobic index value would be substituted with an amino acid with a lesser, or even negative hydrophobicity index. However, substitutions will typically be selected to have minimal adverse effect on other features of protein conformation or function.

Amino acids with hydrophobic side chain that are called aliphatic amino acids will most typically be targeted for substitutions. Examples of this class include alanine, leucine, isoleucine, valine, e.g., those with higher hydrophobicity indices. Other amino acids with hydrophobic side chains like phenylalanine, tryptophan and tyrosine may also be modified or substituted. The substitutions will preferably be with amino acids with electrically charged side chains. Basic examples include arginine, histidine, lysine, while acidic examples include aspartic and glutamic acids. The substitutions presumably would be such that residue changes which affect activity or overall protein conformation are avoided. The residue replacements should also not affect protein structure/function, hence one could apply the standard “conservative” amino acids, such as neutral amino acids. Certain substitutions, e.g., certain histidine or tryptophan replacements, have been observed to enhance salt resistant properties of certain antimicrobial polypeptides. Yu, et al. (2011) Antimicrobial Agents and Chemotherapy 55:4918-921.

Combined with locations of residues for change, the resulting sequence is evaluated for solubility, e.g., using software as described above, to evaluate whether the new sequence is expected to be soluble. For example, the GRAVY score is the average hydropathy score for all the amino acids in the protein, as described above. It is plotted as a red line on the hydropathy plot. According to Kyte and Doolittle (1982), integral membrane proteins typically have higher GRAVY scores than do globular proteins. Though this score is another helpful piece of information, it cannot reliably predict the structure without the help of hydropathy plots such as positive GRAVY (hydrophobic), negative GRAVY (hydrophilic). GRAVY simply calculates overall hydrophobicity of the linear polypeptide sequence with increasing positive score indicating greater hydrophobicity, but no account is taken of the way the protein folds in three dimensions or the percentage of residues buried in the hydrophobic core of the protein.

The entire amino acid sequence of any protein molecule can be taken and one can determine the GRAVY score. If the GRAVY score is low, then one may take only the hydrophobic segment, evaluate the GRAVY score of that segment, and evaluate the effect of substitutions on the total GRAVY score. If there are two or more transmembrane segments, one would focus on with highest GRAVY scores which are predicted to affect solubility, e.g., which have peaks characteristic of insoluble proteins. The threshold GRAVY score would generally be in the range of about −0.5 to +2.0, and higher scores normally need to be lowered while lower scores generally do not affect solubility. One need not always have a negative GRAVY score for a substituted transmembrane segment, as a significant reduction in the average GRAVY score could render the molecule soluble.

Luan, et al. (2004) Genome Res 14(10B):2102-2110 tested the soluble expression of 10,167 full-length C. elegans ORFs and found that protein hydrophobicity was an important factor for an ORF to yield a soluble expression product.

A number of different hydrophobicity scales are available. See, e.g., Eisenberg, et al. (1984) Ann Rev Biochem. 53:595-623; Kallol, et al. (2003) J. Chromatography A 1000:637-655; Rose, et al. (1985) Science 229:834-838. There are some differences between the four scales shown in Table 1. Both the second and fourth scales place cysteine as the most hydrophobic residue, unlike the other two scales which places Ile as the most hydrophobic amino acid. Such a difference apparently could be due to the different methods used to measure hydrophobicity. The Janin (1979) and Rose, et al. (1985) scales examined proteins with known 3-D structures and define the hydrophobic character as the tendency for a residue to be found inside of a protein rather than on its surface and cysteine forms disulfide bonds that must occur inside a globular structure. This may explain why it is ranked as the most hydrophobic amino acids amongst all by these groups. The first and third scales are derived from the physiochemical properties of the amino acid side chains.

The amino acids that are to be selected for mutagenesis for rendering solubility would preferably be from the region of the transmembrane segment. However, if the GRAVY score is not sufficiently reduced after mutation, one could also mutate the amino acid residues that are hydrophobic and close to the postulated transmembrane segment.

Upon design of the variant construct sequence, the sequence is produced. It may be done by synthetic chemical methods, or more preferably by recombinant methods, e.g., site directed mutagenesis of a similar or corresponding first sequence. An appropriate nucleic acid is generated encoding the desired sequence, typically incorporated into an inducible expression vector, and the protein produced, e.g., in the high level prokaryotic expression system. The protein product is then evaluated empirically to confirm that the variant construct is actually produced in soluble form.

In some embodiments, the physicochemical property of protein solubility is the primary desired outcome. This may be applicable where the solubility of the protein product is most important. In other embodiments, the protein product has a biological activity, and the function may also be important to be conserved, an additional limitation to the solubility question. In such circumstances, there may be limitations as to how many and what substitutions are compatible with retention of biological activity, and a minimal number of changes may be preferred. Thus, after a soluble variant incorporating a number of changes is determined to be successful, it may be desired to determine the minimal number of variations which can achieve the desired change in the solubility property. In such a case, individual changes may be changed back to the initial sequence to see whether the solubility is highly dependent upon a particular change. In certain cases, many fewer than the initial proposed changes may suffice to achieve aqueous solubility, and the return of residues to an unmodified sequence is more likely to minimize effect on biological function or minimize antigenic disparity from the first sequence.

One screen is to determine which constructs are produced by the production cell hosts, e.g., that the producing hosts do not kill themselves by expression of the construct. If the cells do not kill themselves upon expression, the protein is not reaching the periplasmic space and the peptidoglycan substrate. Among the constructs which pass that screen, the functional activity screens can be optimized to select for those which retain appropriate balances of membrane translocation activity, catalytic activity, and protein yields.

For proteins which do not possess short hydrophobic transmembrane segments, one could calculate the GRAVY score, identify the hydrophobic amino acid and its hydroplot score, substitute with a most appropriate amino acid that is hydrophilic in nature and the substitution that dramatically reduces the GRAVY score towards the negative value will be adopted. One can determine the hydrophobic residues that project towards the surface, e.g., outside of the protein towards the surrounding solution, using various surface analysis software tools, and seek to decrease the local peak hydrophobicity measures. Typically a localized evaluation, e.g., DAS or local GRAVY measure of the hydrophobic region, is most useful and best comparable across proteins.

The amino acid residues present on the surface of a protein are important in its interaction with other molecules and the solvent, and determine many physical properties, including the structure of the folded protein. In the absence of a 3-D structure, e.g., by crystal structure, the ability to predict surface accessibility of amino acids directly from the sequence is a valuable tool in choosing sites of modification or specific mutations. Prediction of surface exposed residues can be done using several approaches.

One widely used method is by determining the accessible surface area (ASA) or solvent-accessible surface. ASA is the surface area of a biomolecule that is accessible to a solvent. ASA was first described by Lee and Richards. See Lee and Richards (1971) “The interpretation of protein structures: estimation of static accessibility” J. Mol. Biol. 55:379-400. Solvent exposure of amino acids measures how deep residues are buried in tertiary structure of proteins, and hence it provides important information for analyzing and predicting protein structure and functions. See Li, et al. (2011) “QSE: A new 3-D solvent exposure measure for the analysis of protein structure” Proteomics 11:3793-801; and Ahmad, et al. (2003) “Real value prediction of solvent accessibility from amino acid sequence” Proteins 50:629-35.

Another approach is methods based on neural networks for prediction of surface exposed residues. Data from protein crystal structures are used to teach computer-simulated neural networks rules for predicting surface exposure from sequence. These trained networks are able to correctly predict surface exposure. See, e.g., Holbrook, et al. (1990) “Predicting surface exposure of amino acids from protein sequences” Protein Eng. 3:659-665; Rost and Sander (1994) “Conservation and prediction of solvent accessibility in protein families” Proteins 20:216-226; Lebeda, et al. (1998) “Accuracy of secondary structures and solvent accessibility predictions for a clostridial neurotoxin C fragment” J. Protein Chem. 17:311-318; Pollastri, et al. (2002) “Prediction of coordination number and relative solvent accessibility in proteins” Proteins 47:142-153; and Ahmad and Gromiha (2002) “NETASA: neural network based prediction of solvent accessibility” Bioinformatics 18:819-824. Other approaches include logistic function (Mucchielli-Giorgi, et al. (1999) “PredAcc: prediction of solvent accessibility” Bioinformatics 15:176-177); Bayersian analysis (Mucchielli-Giorgi, et al. (1999) “PredAcc: prediction of solvent accessibility” Bioinformatics 15:176-177); information theory (Naderi-Manesh, et al. (2001) “Prediction of protein surface accessibility with information theory” Proteins 42:452-459; Richardson and Barlow (1999) “The bottom line for prediction of residue solvent accessibility” Protein Eng. 12:1051-1054; and Carugo (2000) “Prediction residue solvent accessibility from protein sequence by considering the sequence environment” Protein Eng. 13:607-609); and substitution matrices (Pascarella, et al. (1998) “Easy method to predict solvent accessibility from multiple sequence alignments” Proteins 32:190-199). A less quantitative approach to predict solvent accessibility is simply based on hydrophobicity plots (see Lesk (2002) Introduction to Bioinformatics Oxford University Press).

Surface Residue Prediction Tools:

InterProSurf: Protein-Protein Interaction Server. This provides the functions to predict interacting residues on a monomeric protein surface and to find or identify interface residues in a protein complex. The number of surface atoms are given and visualized on the basis of top five clusters and the next five clusters. See the website available at curie.utmb.edu/prosurf.html.

SPPIDER, Solvent accessibility based Protein-Protein Interface Identification and Recognition” tools. These provide a representation which integrates enhanced relative solvent accessibility (RSA) predictions with high resolution structural data. RSA prediction-based fingerprints of protein interactions significantly improve the discrimination between interacting and noninteracting sites. See the website available at sppider.cchmc.org.

PPI-pred, PPI-Pred predicts protein-protein binding sites using a combination of surface patch analysis and a support vector machine (SVM). It will take any type of protein in PDB format as input, and the output identifies the most likely binding site location and two other possible locations. It calculates properties over the protein surface likely to distinguish protein-protein binding sites from the rest of the surface: using, e.g., hydrophobicity, residue interface propensity, electrostatic potential, solvent accessible surface area, surface topography (shape), and sequence conservation. See the website available at bmbpcu36.leeds.ac.uk/ppi_pred/overview.html.

meta-PPISP. meta-PPISP is built on three individual web servers: cons-PPISP, PINUP, and Promate. The system uses a linear regression method, using the raw scores of the three severs as input. Cross validation showed that meta-PPISP outperforms all the three individual servers. See the website available at pipe.scs.fsu.edu/meta-ppisp.html.

For proteins with no clear transmembrane segments, one would apply structure modeling of the gene of interest to determine surface exposed amino acid residues and their hydrophobicity index. If the hydrophobicity index or the GRAVY score is on the negative side then replacing the less hydrophilic moieties with higher hydrophilic residues might achieve higher soluble protein.

The various methods that have been developed allow prediction of the accessibility status (exposed, buried, and, possibly, intermediate) of each residue with reasonably high accuracy. The residues which are exposed to the solvent are more likely to affect solubility of the protein and its interaction with the polar water solvent. These are the residues which are most likely to positively affect solubility when substituted with a more polar or hydrophilic residue.

Such substitutions need not be conservative substitutions and could be selected to evaluate the differential effects on reduction of the hydrophobicity index; thereafter screening would be performed to determine the effect of such changes on solubility of the expressed protein along with functionality.

Recombinant proteins expressed in Pichia pastoris is intended to result in soluble proteins in the extracellular medium. Hydrophobic interaction may play a crucial role in bioactivity of proteins and it is not universally true that all soluble proteins are expected to be in right conformation. Bahrami et al. (2009) reported such in the expression of recombinant human granulocyte colony stimulating factor (rhG-CSF) in the methylotropic yeast Pichia pastoris under the control of the AOX1 promoter. See Bahrami, et al. (2009) “Prevention of human granulocyte colony-stimulating factor protein aggregation in recombinant Pichia pastoris fed-batch fermentation using additives” Biotechnol. Applied Biochem. 52:141-148. This host yielded a maximum concentration of 0.6 mg rhG-CSF g-methanol −1 as a soluble protein, however, the secreted rhG-CSF was shown to exist as aggregates in the culture broth due to hydrophobic interaction. To prevent undesirable protein aggregation, the effect of additional additives in P. pastoris culture medium were investigated. Among 7 additives tested, Tween20, Tween80, and betain exhibited the best results in preventing the formation of rhG-CSF protein aggregates. Similar results have been reported for interferon alpha mutant when expressed in Pichia pastoris. Wu, et al. (2008) “Inhibition of degradation and aggregation of recombinant human consensus interferon-α mutant expressed in Pichia pastoris with complex medium in bioreactor” Appl. Microbiol. Biotechnol. 80:1063-1071. Thus, the methodology of hydrophobicity change may be applicable to different production systems, and may be useful in contexts where changes in the hydrophobicity of protein may affect ability to resolubilize or refold into active conformation.

The changes in hydrophobicity may be combined with other strategies, e.g., applicable to situations where insolubility is partly also attributable to disulfide mispairing. Reteplase is a truncated version of the human tissue plasminogen activator (tPA) used in the therapy of myocardial infarction. Due to nine disulphide linkages, the expression of this protein in E. coli is cumbersome since the process involves the denaturation and refolding of the protein. E. coli is the first choice for expression and purification of this protein since the molecule does not require glycosylation for activity. This protein has been successfully expressed in Pichia pastoris in soluble and active state. Mandi, et al. (2010) “Asn12 and Asn278: Critical residues for in-vitro activity of reteplase” Adv. Hematology 2010:172484. Epub 2010 Jun. 21. For proteins which have high content of cysteine residues, a combination of depletion by substitution of cysteine residues content with hydrophobicity value reduction could achieve successful expression levels in E. coli as an active soluble entity.

Two classes of proteins play an important role in in vivo protein folding during protein expression in E. coli. These are use of molecular chaperones like GroEs/GroEL, DnaK-DnaJ-GrpE and ClpB that promote the proper isomerization and cellular targeting by transiently interacting with folding intermediates. Three types of foldases are also known to play an important role in protein folding. These are peptidyl prolyl cis/trans isomerases (PPI's), disulfide oxidoreductase (DsbA) and disulfide isomerase (DsbC) and protein disulfide isomerase (PDI)—an eukaryotic protein that catalyzes both protein cysteine oxidation and disulfide bond isomerization. Co-expression of one or more of these proteins with the target protein could lead to higher levels of soluble protein. The levels of co-expression of the different chaperones/foldases have to be optimized for each individual case. The solubility of disulfide bond containing protein can be increased by using a host strain with a more oxidizing cytoplasmic environment. Two strains are commercially available (Novagen): AD494, which has a mutation in thioredoxin reductase (trxB) and Origami, a double mutant in thioredoxin reductase (trxB) and glutathione reductase (gor).

Proteins that are toxic to E. coli may be expressed in cell lines such as CD43/CD41 DE3. CD43(DE3) is a derivative of BL21(DE3) and was reported to overproduce TM proteins with less toxicity. See Miroux and Walker (1996) J. Mol. Biol. 260:289-98. Keeping protein expression at a moderate level can maximize yields by maintaining the concentration of a toxic target protein just below a host strain's tolerance. Alternatively, tuning expression by selection of appropriate promoter system to prevent well-expressed target proteins from creating inclusion bodies is another strategy. The rhamnose/arabinose/lac/Trc/Trp/lambda/pL promoters are part of many expression systems. In other embodiments, expression of soluble and toxic proteins in a prokaryotic expression system could be made at hyperexpression levels where the protein is insoluble and inactive, e.g., in inclusion bodies, may be a useful strategy. This could be achieved by fusing appropriate lengths of suitable hydrophobic segments at the N or C terminus into the native protein, with or without protease cleavage site, and such a fusion protein could be hydrophobic and hence insoluble in the high expression system. This may prevent toxic interactions of the expressed protein inside the cell.

When disulfide bonds are essential for target protein folding or stability, efforts are made to direct the protein to E. coli's oxidative periplasm, where Dsb enzymes can establish the correct bond configuration. Several commercially available vectors include an N-terminal signal sequence for exporting proteins to the periplasm. Alternatively, New England Biolab's SHuffle strains are excellent options for expressing proteins with complex disulfide bonds. These strains carry mutations that alter cellular reduction conditions, allowing proper disulfide bond formation in a now-partially oxidizing cytoplasm and also express disulfide bond isomerase (DsbC) in the cytoplasm, rather than only in the periplasm of E. coli. These various expression hosts may be combined with the methods and constructs described herein to provide soluble production of appropriate proteins.

There also exist examples of proteins which are essentially not expressible in E. coli, as indicated above. Some of these possess hydrophobic N termini, e.g., enterokinase (EK) has MIVGG as the few amino acids at the N terminus. Interestingly, MIV is highly hydrophobic and possibly changing these residues to hydrophilic residues, the EK gene might get expressed as a soluble entity in E. coli and might retain biological activity.

Methionine aminopeptidase (MetAP) is a ubiquitous enzyme in both prokaryotes and eukaryotes, which catalyzes co-translational removal of N-terminal methionine from elongating polypeptide chains during protein synthesis. It specifically removes the terminal methionine in all organisms, if the penultimate residue (P1′) is non-bulky and uncharged. The extent of removal of methionyl from a protein is dictated by its N-terminal peptide sequence. Earlier studies revealed that MetAPs require amino acids containing small side chains (e.g., Gly, Ala, Ser, Cys, Pro, Thr, and Val) as the P1′ residue, but their specificity at positions P2′ and beyond remains incompletely defined. The catalytic activity of human MetAP2 toward Met-Val peptides is consistently 2 orders of magnitude greater than that of MetAP1, suggesting that MetAP2 is responsible for processing proteins containing N-terminal Met-Val and Met-Thr sequences in vivo. See Xiao, et al. (2010) “Protein N-Terminal Processing: Substrate Specificity of Escherichia coli and Human Methionine Aminopeptidases” Biochemistry 49:5588-5599). At positions P2′-P5′, all three MetAPs have broad specificity but are poorly active toward peptides containing a proline at the P2′ position.

The MAP is also responsible for removal of the N terminal initiation Met in the host cell. As such, when the amino acid is removed, the numbers assigned to particular residues changes accordingly. Thus, in the sequence listings, the product from expression of a defined nucleic acid construct may depend upon the activity of the respective MAPs. In certain circumstances, whether the Met remains or is removed will depend upon the physiology of the cell, the MAP activity, and perhaps other features of the nascent polypeptide. As such, the numbers assigned to particular residues may be off by the amount of processing which occurs to the proteins, and in particular, the actual cellular product forms may lack the N terminal Met.

It is possible that alteration of the N terminal sequence of any protein by changing its hydrophobicity could enhance the chances of removal of the N terminal methionine from the protein being expressed by the activity of the methionine amino peptidase of the host and this would bring about achievement of authentic N terminus of the protein of interest. For this reason, the activity of certain recombinant proteins may be affected by the proper or improper activity of the resident MAP in a producing host cell. For example, perhaps the lack of activity of coli expressed proteins may be attributed to a mechanism such as differential MAP activity. In which case the lack of activity or expression of certain genes will be resolved by modifications to local protein conformation achievable through these techniques.

Since it is customary to conduct clinical trials for new biological molecules, modification of hydrophobicity of therapeutic genes is not usually attempted by clinical researchers. Accordingly such data becomes of pure academic interest. Hence, substituting hydrophobic residues might open opportunities for different diagnostic enzymes or enzymes like cellulases, amylases, hemicellulases, glucosidases, etc., used for detergent industries since strategies to obtain soluble expression of such proteins would be of immense value.

General fundamentals of biotechnology, principles and methods are described, e.g., in Alberts, et al. (2002) Molecular Biology of the Cell (4th ed.) Garland; Lodish, et al. (1999) Molecular Cell Biology (4th ed.) Freeman; Janeway, et al. (eds. 2001) Immunobiology (5th ed.) Garland; Flint, et al. (eds. 1999) Principles of Virology: Molecular Biology, Pathogenesis, and Control, Am. Soc. Microbiol.; Nelson, et al. (2000) Lehninger Principles of Biochemistry (3d ed.) Worth; Freshney (2000) Culture of Animal Cells: A Manual of Basic Technique (4th ed.) Wiley-Liss; Arias and Stewart (2002) Molecular Principles of Animal Development, Oxford University Press; Griffiths, et al. (2000) An Introduction to Genetic Analysis (7th ed.) Freeman; Kierszenbaum (2001) Histology and Cell Biology, Mosby; Weaver (2001) Molecular Biology (2d ed.) McGraw-Hill; Barker (1998) At the Bench: A Laboratory Navigator CSH Laboratory; Branden and Tooze (1999) Introduction to Protein Structure (2d ed.), Garland Publishing; Sambrook and Russell (2001) Molecular Cloning: A Laboratory Manual (3 vol., 3d ed.), CSH Lab. Press; Scopes (1994) Protein Purification: Principles and Practice (3d ed.) Springer Verlag; Simpson, et al. (eds. 2009) Basic Methods in Protein Purification and Analysis: A Laboratory Manual CSHL Press, NY, ISBN 978-087969868-3; Friedmann and Rossi (eds. 2007) Gene Transfer: Delivery and Expression of DNA and RNA, A Laboratory Manual CSHL Press, NY, ISBN 978-087969764-8; Link and LaBaer (2009) Proteomics: A Cold Spring Harbor Laboratory Course Manual CSHL Press, NY, ISBN 978-087969793-8; and Simpson (2003) Proteins and Proteomics: A Laboratory Manual CSHL Press, NY, ISBN 978-087969554-5. Other references directed to bioinformatics include, e.g., Mount (2004) Bioinformatics: Sequence and Genome Analysis (2d ed.) CSHL Press, NY, ISBN 978-087969687-0; Pevsner (2009) Bioinformatics and Functional Genomics (2d ed.) Wiley-Blackwell, ISBN-10: 0470085851, ISBN-13: 978-0470085851; Lesk (2008) Introduction to Bioinformatics (3d ed.) Oxford Univ. Press, ISBN-10: 9780199208043, ISBN-13: 978-0199208043; Zvelebil and Baum (2007) Understanding Bioinformatics Garland Science, ISBN-10: 0815340249, ISBN-13: 978-0815340249; Baxevanis and Ouellette (eds. 2004) Bioinformatics: A Practical Guide to the Analysis of Genes and Proteins (3d ed.) Wiley-Interscience; ISBN-10: 0471478784, ISBN-13: 978-0471478782; Gu and Bourne (eds. 2009) Structural Bioinformatics (2d ed., Wiley-Blackwell, ISBN-10: 0470181052, ISBN-13: 978-0470181058; Selzer, et al. (2008) Applied Bioinformatics: An Introduction Springer, ISBN-10: 9783540727996, ISBN-13: 978-3540727996; Campbell and Heyer (2006) Discovering Genomics, Proteomics and Bioinformatics (2d ed.), Benjamin Cummings, ISBN-10: 9780805382198, ISBN-13: 978-0805382198; Jin Xiong (2006) Essential Bioinformatics Cambridge Univ. Press, ISBN-10: 0521600820, ISBN-13: 978-0521600828; Krane and Raymer (2002) Fundamental Concepts of Bioinformatics Benjamin Cummings, ISBN-10: 9780805346336, ISBN-13: 978-0805346336; He and Petoukhov (2011) Mathematics of Bioinformatics: Theory, Methods and Applications (Wiley Series in Bioinformatics), Wiley-Interscience, ISBN-10: 9780470404430, ISBN-13: 978-0470404430; Alterovitz and Ramoni (2011) Knowledge-Based Bioinformatics: From analysis to interpretation Wiley, ISBN-10: 9780470748312, ISBN-13: 978-0470748312; Gopakumar (2011) Bioinformatics: Sequence and Structural Analysis Alpha Science Intl Ltd., ISBN-10: 184265490X, ISBN-13: 978-1842654903; Barnes (ed. 2007) Bioinformatics for Geneticists: A Bioinformatics Primer for the Analysis of Genetic Data (2d ed.) Wiley, ISBN-10: 9780470026199, ISBN-13: 978-0470026199; Neapolitan (2007)Probabilistic Methods for Bioinformatics Kaufmann Publishers, ISBN-10: 0123704766, ISBN-13: 978-0123704764; Rangwala and Karypis (2010) Introduction to Protein Structure Prediction: Methods and Algorithms (Wiley Series in Bioinformatics), Wiley, ISBN-10: 0470470593, ISBN-13: 978-0470470596; Ussery, et al. (2010) Computing for Comparative Microbial Genomics: Bioinformatics for Microbiologists (Computational Biology), Springer, ISBN-10: 9781849967631, ISBN-13: 978-1849967631; and Keith (ed. 2008) Bioinformatics: Volume I: Data, Sequence Analysis and Evolution (Methods in Molecular Biology), Humana Press, ISBN-10: 9781588297075, ISBN-13: 978-1588297075.

The following discussion is for the purposes of illustration and description, and is not intended to limit the invention to the form or forms disclosed herein. Although the description has included description of one or more embodiments and certain variations and modifications, other variations and modifications are within the scope of the invention, e.g., as may be within the skill and knowledge of those in the art, after understanding the present disclosure. All publications, patents, patent applications, Genbank numbers, and websites cited herein are hereby incorporated by reference in their entireties for all purposes.

### EXAMPLES

### Example 1

### P271 (P266) Construct and Biological Activity

An alternative construct of the P225 construct was designed encoding a protein. See SEQ ID NO: 4; nucleic acid construct is SEQ ID NO: 3. The N terminal Met will typically be removed in a prokaryotic host due to the action of host methionine amino peptidase that effectively removes N terminal methionine leaving a protein beginning with the penultimate amino acid namely Gly in this case. The N-proximal His segment was shortened to 6 His, and a segment of following histidine amino acids was deleted. This provided a construct having segments: 6×His tag-GP36 CD-RRR-BPI TMD-RRR. The GP36 CD would run from about Gly(9) to Glu(224), the first RRR corresponds to R(225) to R(227), the BPI TMD corresponds to Ala(228) to R(251), and the final RRR corresponds to residues 252-254. The projected molecular weight of the computed translation should be about 27.6 kDa, with a theoretical pI of about 9.48. This includes the N terminal Met, which is generally removed.

Like the P225 construct, the protein was found to be insoluble upon expression in E. coli BL21 (DE3) cells after induction with IPTG. Briefly, inclusion bodies (IB) were isolated, the pellet solubilized in 6M GuHCl, purified on a Ni-NTA affinity column under denaturing conditions and the protein eluted in 8M urea.

In more detail, the induced cell pellet was resuspended in lysis buffer (50 mM Tris base, 0.1M NaCl, 0.1% TritonX100), and sonicated using a 13 mm probe for 10 minutes. The sonicated cell pellet was centrifuged at 16,000 rpm for 10 minutes and the inclusion bodies pellet collected. The inclusion body pellet was solublized by resuspending the pellet in Buffer A (6M GuHCl, 100 mM NaH₂PO₄, 10 mM TrisCl, pH 8.0) and kept rocking for 30 min at room temperature. The ratio of IB: buffer volume was 1 gram wet weight of IB with 40 ml of buffer A. The solubilized proteins were centrifuged at 16,000 rpm for 10 min and the clear supernatant was collected. Ni-NTA matrix was equilibrated with Buffer B (8M urea, 100 mM NaH₂PO₄, 10 mM TrisCl, pH 8.0) with 5 column volumes used for equilibration. The solubilized clear supernatant was loaded on to the equilibrated Ni-NTA column and allowed to pass through in gravity mode and the flow through collected. The column was washed with 10 column volumes of Buffer B to remove impurities and unbound proteins. It was then washed with 10-15 column volumes of Buffer C (8M urea, 100 mM NaH₂PO₄, 10 mM TrisCl, pH 6.5). The protein elutions were carried out in Buffer E (8M urea, 100 mM NaH₂PO₄, 10 mM TrisCl, pH 4.5). Fractions were collected and analyzed by SDS PAGE. Fractions containing protein of interest in high amounts as seen on SDS PAGE gels were pooled and dialyzed in a stepwise manner. Dialysis was carried out against a buffer volume ˜100 times of the pooled eluate volume (e.g., 10 ml eluate dialized against 1 liter buffer), in three steps, first against 4M Urea in 20 mM sodium phosphate buffer, pH 6.0, for 5 hrs at 4 deg C.; second against 2M urea in 20 mM sodium phosphate buffer, pH 6.0, for 5 hrs at 4 deg C.; and third against 20 mM sodium phosphate buffer, pH 6.0, with 5% sucrose, 5% sorbitol, and 0.2% Tween 80, for 5 hrs at 4 deg C. Eluates taken out post dialysis were centrifuged to separate any precipitation. The cleared supernatant was collected and protein content estimated for activity assay.

The sucrose, sorbitol, and Tween80 components help stabilize the protein from aggregation and precipitation. The final product was about 85-95% homogeneous by SDS PAGE with coomassie blue staining and silver staining.

The structure of the protein is as follows:

SEQ ID NO: 1 P271 (P266) construct Nucleic acid:
1-6 = ATG (start codon) GGC: Bases generated due to cloning
enzyme (NheI) site
7-24 = Sequence encoding 6Xhis tag
25-672 = Sequence encoding GP36CD sequence
673-681 = Sequence encoding linker arginines
682-753 = Sequence encoding BPI MTD
754-762 = Sequence encoding terminal arginines
763-765 = TGA: Sequence encoding stop codon
SEQ ID NO: 2 P271 (P266) amino acid sequence (254 aa):
1 = M (start codon; removed by producing coli host)
2 = G: Amino acid generated due to cloning enzyme site
3-8 = 6Xhis tag
9-240 = GP36 Catalytic (muralytic) Domain sequence
241-243 = Linker arginines
228-251 = BPI TMD
252-254 = N-Terminal arginines

The purified protein was assayed for bacterial killing using a CFU drop assay and typically simultaneously monitored for residual OD600 at the end of 16 hours of treatment with the protein product. Log phase PA01 Pseudomonas aeruginosa target cells were resuspended in a suitable buffer at an absorbance of 1.0, which corresponds to about 1E7 cells. The protein was tested at 50 μg in either acetate or glycine buffers. The assays were performed in 20 mM sodium phosphate buffer (pH 6.0), 5% sucrose, 5% sorbitol, and 0.2% Tween80 with either 20 mM sodium acetate (pH 6.0) or 50 mM glycine-NaOH (pH 7.0) at 37° C. for 2 hrs at 200 rpm agitation.

The CFU drop assay in sodium acetate buffer provided about 5 logs drop, and in the glycine buffer provided at least 7 logs drop after treatment with the protein. From the residual OD600, the acetate buffer provided about 80% less in comparison to control, while the glycine buffer provided about 95% residual decrease in comparison to control.

The CFU drop assay in glycine buffer (pH 7.0) was evaluated without the sucrose, sorbitol, and tween80 stabilizers in the incubation. The CFU drop without stabilizers was the same with stabilizers in the assay, at least 7 logs drop. In many cases, other stabilizers or additives may be useful or important. These may include materials such as polyols, e.g., sorbitol and related compounds; glycerols, e.g., in the range of 0-10%; sugars, such as sucrose, e.g., in the range of 0-5%; detergents or surfactants such as Triton X100, Brij 35, NP-40, Tween 20, Octylbetaglucoside, Sarkosyl, Tween80, etc., preferably tween80, e.g., in the range of 0.1% to 0.5%; and metal chelators such as EGTA, EDTA, preferably EDTA, e.g., in the range if 50 μM-100 μM.

The biological activity of P271 (P266 has the same polypeptide sequence, but is encoded on a different plasmid) was titrated across protein concentration on the PA01 target strain. Both the CFU drop and the residual OD600 progressed with 2 hr incubations as the protein was increased from 5, 10, 25, and 50 μg protein. Under the conditions tested, both by CFU drop and residual OD600, with 50 μg P266 at 37° C. and 2 hr incubation, treatment could kill virtually all cells at 1E6 and 1E7 cells in the assay, but showed much decreased killing with 1E8 or more cells in the assay. Incubation time over the 1-4 hour range did not seem to have dramatic effects on PA01 killing assays.

Testing stability of P271 (P266) at various temperatures, the protein maintained killing activity after 1 hr exposure to 37, 42, and 65° C. The product is heat stable up to 65° C. for an hour.

Testing target killing efficiency, P271 (P266) had substantial killing activity, by both the CFU drop and OD600 drop assays, on Pseudomonas aeruginosa, NDM1 plasmid carrying Klebsiella pneumoniae, NDM1 plasmid carrying E. coli, Klebsiella pneumoniae, Acinetobacter baumanii, Salmonella typhimurium, Salmonella infantis, and E. coli isolates. Similar assays indicated some but lesser activity on Shigella, Proteus mirabilis, and Burkholderia thailandensis isolates, but conditions were not optimized to determine quantitative measures. Similarly, activity on Gram-positive isolates were not high, but would likely be detected with greater amounts of protein, longer incubation times, fewer cells, or modification of other parameters. Thus, P271 (P266) has quite broad target bacteria species activity. This is broader than known phage infection specificity, though the catalytic domain used is derived from a gram negative phage Pseudomonas aeruginosa virion expressed structure.

The effect of P271 (P266) incubation with human red blood cells was minimal at the highest tested 25 and 50 μg amounts. With 1 hr incubations, the red blood cells maintained integrity, e.g., containing hemoglobin, and the cells could be sedimented into pellets. This indicates the protein does not disrupt eukaryotic cell membranes, and allows for therapeutic uses of this protein product.

### Example 2

### Soluble P271 (P266) Variant; P275

The P271 (P266) protein can be difficult to handle, as it can be insoluble. This makes its production in prokaryotic expression hosts difficult, as the protein precipitates into inclusion bodies. This insolubility requires the protein purification to solubilize the protein from the inclusion bodies, typically in denatured form, with Guanidinium HCl and urea and refolding which may lead to significant losses of protein into inactive conformation forms. In addition, protein oxidation increases the hydrophobicity contributing to further losses in activity, along with protein instability and aggregation, e.g., due to adsorption to apparatus and container surfaces used in the purification processes.

Partly also to determine whether variations in the sequence of the MTD domain retain activity, a variant was designed which might decrease the local hydrophobicity in the BPI segment. This was attempted also in part to subtly disrupt the folded structure of the protein to expose more of the hydrophobic interior to the aqueous solution. This might also dehydrate the shells of water molecules that form over the hydrophobic patches on the surface of properly folded proteins.

In particular, a nucleic acid construct was designed to generate a variant protein from the P266, designated P275, with conversions of V232 to E; V234 to D; and 1236 to K. See SEQ ID NO: 3 and 4.

This construct produced a product which exhibited a number of surprising and unexpected properties. The expression construct was expressed in E. coli BL21(DE3) with induction at 37° C., 1 mM IPTG, as was the P266 expression. However, the P275 did not form inclusion bodies, and the majority of the protein product was restricted to the soluble fraction. Quite unexpectedly, the variant did not precipitate into inclusion bodies during culture. Moreover, the soluble protein did not traverse the bacterial cell membrane to access the peptidoglycan layer (located in the periplasmic space) to kill the Gram-negative E. coli production cell host. Thus, there exists with these MTD constructs the possibility of maintaining sufficient intracellular solubility without the MTD providing the protein function of traversing the bacterial cell membrane. However, the MTD retains the function of allowing the construct to traverse the outer cell wall, thereby providing the protein construct access (across the outer cell wall into the periplasmic space) to the sensitive peptidoglyan layer otherwise protected by that outer cell wall of the Gram-negative bacteria.

Remaining a soluble protein, the P275 product was much simpler to handle in purification and recovery, and provided much higher yields of active protein. The soluble P275 protein was purified on the Ni-NTA column at pH 8.0; eluted with imidazole at pH 4.5, dialyzed to remove imidazole, and reformulated into assay buffer.

The P275 induced cell pellet was resuspended in Lysis buffer (50 mM Tris Base, 0.1M NaCl, 0.1% TritonX100) and sonicated. The sonicated cell pellet was centrifuged 16,000 rpm for 10 min, and the supernatant collected and pH adjusted to 8.0. A Ni-NTA matrix was equilibrated with (50 mM Tris.Cl, pH 8.0) using 5 column volumes. The solubilized protein was loaded on to the equilibrated Ni-NTA column and allowed to pass through. The flow through was collected and passed through the column once again. The column was washed with 10-15 column volumes of 20 mM sodium phosphate buffer, pH 6.5, then washed with 5 column volumes of 20 mM sodium phosphate buffer, pH 4.5. Protein elution was carried with 1M imidazole in 20 mM sodium phosphate buffer, pH 4.5. Eluted fractions were collected and analyzed by SDS PAGE. Fractions containing the protein of interest in high amounts as seen on SDS PAGE gels were pooled and dialyzed. Dialysis was carried out against a buffer volume 100 times of the pooled eluate volume, three changes against 20 mM sodium phosphate buffer, pH 6.0 each for 5 hrs at 4 deg C. Eluates taken out post dialysis were centrifuged to separate any precipitation, and the supernatant collected and additives sucrose, sorbitol, and Tween80 were added to a final concentration of 5%, 5%, and 0.2% respectively. Protein content was estimated for activity assays.

The P275 product is soluble and easy to purify, which allows a more cost effective downstream operation avoiding the requirement for denaturing agents, and achieving about 85% purity in a simple process leading to a biologically active product.

The P275 product exhibits a comparable or better CFU drop assay under standard 50 μg protein amounts at 37° C. with 2 hr incubation times.

### Example 3

### Expression, Purification, and Testing of New Constructs

The described methods are exemplary, and can be modified to particular equipment or preferences. Thus, the concentrations, times, buffers, media, and such may be modified and might provide essentially equivalent results. Thus, different length or composition linker segments may often be substituted, or the boundaries of domains modified to exclude or include additional flanking sequence.

A. Expression of Above Constructs

Each the above constructs could be optimized for expression by choosing the best codons for expression in E. coli (codon bias), changing the GC content, incorporating alternate fusion tags (e.g., glutathione S-transferase GST), nusA transcription elongation factor, maltose binding protein (MBP), intein, among many possibilities), varying inducer concentrations, temperature, expression with chaperones to help in better folding and choosing different expression hosts. Loss of biological activity is a most sensitive measure of incorrect protein conformation, and a low specific activity of a protein preparation may be an indicator that much of the protein is not folded correctly.

B. Expression

Competent cells of appropriate expression host, e.g., E. coli, are transformed with the respective plasmid, plated on LB+ampicillin (100 μg/ml) or kanamycin (20 μg/ml), and incubated overnight at 37 deg C. The cultures from plates are scraped into LB+antibiotic, typically liquid, and grown to OD₆₀₀˜0.8 to 1.0. The cells are then induced with IPTG at 1 mM and incubated at 37 deg C. for 4 hours. The cells are harvested by centrifugation at 8000 rpm for 10 minutes and the pellet stored at −80 deg C.

C. Product Purification

In many cases, the constructs may accumulate in inclusion bodies. The induced cell pellet is resuspended in lysis buffer (50 mM Tris base, 0.1 M NaCl, 0.1% TritonX100), and sonicated using a 13 mm probe for 10 minutes. The sonicated cell pellet is centrifuged at 16,000 rpm for 10 minutes and a pellet containing inclusion bodies (IB) is collected. The inclusion body pellet is solubilized by resuspending the pellet in Buffer A (6M GuHCl, 100 mM NaH₂PO₄, 10 mM TrisCl, pH 8.0) and kept rocking for 30 mins at room temperature. The ratio of IB: buffer volume is typically 1 gram wet weight of IB with 40 ml of buffer A. The lysate is centrifuged at 16,000 rpm for 10 min and the clear supernatant is collected. A Ni-NTA matrix is equilibrated with Buffer B (8M urea, 100 mM NaH₂PO₄, 10 mM TrisCl, pH 8.0) with 5 column volumes used for equilibration. The supernatant from the IB is loaded on to the equilibrated Ni-NTA column and allowed to pass through in gravity mode and the flow through is collected. The column is washed with 10 column volumes of Buffer B to remove impurities and unbound proteins. The column is then washed with 10-15 column volumes of Buffer C (8M urea, 100 mM NaH₂PO₄, 10 mM TrisCl, pH 6.5). The attached protein elutions are carried out in Buffer E (8M urea, 100 mM NaH₂PO₄, 10 mM TrisCl, pH 4.5). Fractions are collected and analyzed by SDS PAGE. Fractions containing protein of interest in high amounts as seen on SDS PAGE gels are pooled and dialyzed in a stepwise manner. The pooled fractions are subject to dialysis carried out against a buffer volume ˜100 times of the pooled eluate volume (e.g., 10 ml eluate dialized against 1 liter buffer). The dialysis is performed first against 4M urea in 20 mM sodium phosphate buffer, pH 6.0, for 5 hrs at 4 deg C.; then secondly against 2M urea in 20 mM sodium phosphate buffer, pH 6.0, 5 hrs at 4 deg C.; and thirdly against 20 mM sodium phosphate buffer, pH 6.0 with 5% sucrose, 5% sorbitol, and 0.2% tween80 for 5 hrs at 4 deg C. Eluates taken out post dialysis are centrifuged to separate any precipitated material. The cleared supernatant is collected and protein content estimated for activity assay.

D. Assays

The P271 (P266) and P275 protein constructs were produced to exhibit antimicrobial activity, or target cell killing. A CFU drop assay is typically performed essentially as follows. Bacterial cells are grown in LB broth to absorbance at 600 nm reaches a range of 0.8 to 1.0. Then 1 ml of culture is spun at 13000 rpm for 1 minute and supernatant discarded. The cell pellet is resuspended in one ml of 50 mM Glycine-NaOH buffer (pH 7.0) and cell numbers adjusted to about 1×10⁸/ml. Test protein is added to 100 μl cells to achieve final concentration of about 50 μg and volume made-up to 200 μl with 20 mM sodium phosphate buffer (pH 6.0) with additives. The protein is incubated with cells at 37 deg C. for 2 hours with 200 rpm agitation, then the samples are log diluted in LB broth and plated on LB agar to quantitate residual CFU. The plates are incubated at 37 deg C. overnight for colonies to grow.

An alternative Metabolic Dye Reduction assay can determine live cell numbers. The assay is based on the principle that viable cells reduce Iodo-Nitro Tetrazolium (INT), a metabolic indicator dye. Briefly, 1×10⁷ target cells, e.g., P. aeruginosa, in 100 μA volume are mixed with test protein in 100 μl to achieve final concentration of about 50 μg and volume made-up to 200 μA with 20 mM sodium phosphate buffer (pH 6.0) with additives in microtiter plate wells. A cell control is also maintained. Samples are incubated at 37 deg C. with 200 rpm for 2 hour and INT dye (1×) is added to all samples. The microplate is incubated in dark at room temperature for 20 minutes and the absorbance at 492 nm is recorded. 10×INT stock solutions are prepared by dissolving 30 mg Tetrazolium Violet (Loba Chemie, India) in 10 ml of 50 mM Sodium Phosphate buffer, pH 7.5.

### Example 4

### Binding Studies

The P271 (P266) and P275 antimicrobial proteins have a hydrolytic activity which acts on the proteoglycan layer of its target bacteria. In Gram-negative bacteria, this substrate is sequestered from the external solution by the Outer Membrane, which prevents normal proteins from binding to the peptidoglycan substrate. Thus, whether the protein binds to the substrate is a surrogate measure of the activity and proper conformation of the protein.

In Gram-negative bacteria, the outer membrane and the peptidoglycan are linked to each other with lipoproteins, and the OM includes porins, which allow the passage of small hydrophilic molecules. See, e.g., Cabeen and Jacobs-Wagner (2005) “Bacterial Cell Shape” Nature Revs. Microbiology 3:601-610; Nikaido (2003) “Molecular basis of bacterial outer membrane permeability revisited” Microbiol. Mol. Biol. Rev. 67:593-656. The structure and composition of the outermost layer of the cells is reported to be different between different bacteria. On the outer envelope cells may have polysaccharide capsules (see, e.g., Sutherland (1999) “Microbial polysaccharide products” Biotechnol. Genet. Eng. Rev. 16:217-29; and Snyder, et al. (2006) “Structure of a capsular polysaccharide isolated from Salmonella enteritidis” Carbohydr. Res. 341:2388-97.) or protein S-layers (Antikainen, et al. (2002) “Domains in the S-layer protein CbsA of Lactobacillus crispatus involved in adherence to collagens, laminin and lipoteichoic acids and in self-assembly” Mol. Microbiol. 46:381-94; Schäffer and Messner (2005) “The structure of secondary cell wall polymers: how Gram-positive bacteria stick their cell walls together” Microbiology. 151:643-51; and Avall-Jääskeläinen and Palva (2005) “Lactobacillus surface layers and their applications” FEMS Microbiol Rev. 29:511-29), which protect bacteria in unfavorable conditions and affect their adhesion. The basic structure of lipopolysaccharide (LPS), a covalently linked lipid and heteropolysaccharide, is common to all LPS molecules studied, but there are extensive variations in the chemical structures of LPS depending on bacterial genera, species, and strains. See, e.g., Trent, et al. (2006) “Diversity of endotoxin and its impact on pathogenesis” J. Endotoxin Res. 12:205-23; Raetz and Whitfield (2002) “Lipopolysaccharide endotoxins” Ann. Rev. Biochem. 71:635-700; Yethon and Whitfield (2001) “.Lipopolysaccharide as a target for the development of novel therapeutics in gram-negative bacteria” Curr. Drug Targets Infect. Disord. 1:91-106; and Yethon and Whitfield (2001) “Purification and characterization of WaaP from Escherichia coli, a lipopolysaccharide kinase essential for outer membrane stability” J. Biol. Chem. 276:5498-504. Hence, binding studies appear very relevant for testing the efficacy of the anti-bacterial agent.

Thus, some assay may be used to determine whether the construct can reach the enzyme substrate, or is sticking to extraneous surfaces or materials. Described here are various surrogate assays for whether the construct (with MTD) reaches the peptidoglycan layer.

A first assay is SDS-PAGE for checking the binding or absorption of the protein to cells. For example, 10⁷ cells are treated with a suitable amount of protein for approximately 2 hours. Then the cells are pelleted by centrifugation and the amount of protein in the supernatant is examined on SDS-PAGE and stained. The protein is labeled as adsorbed to cells, if the intensity of the protein before the adsorption to cells is higher than the one after adsorption, the difference is likely to be due to cell binding.

A second assay is confocal imaging to demonstrate/visualize bacterial outer membrane changes upon protein binding. A third assay is to link to the protein to fluorescent tags for examining the fluorescence upon protein binding to substrate structures. A fourth assay is to determine the leakage of cellular contents by luciferase based assay.

### Example 5

### Target Residues for P134 Holin Sequence

The fusion of a GP36CD-P134holin protein is described in SEQ ID NO:5. The residues which are indicated for replacement to generate a more soluble variant are:

Ala249, Val250, Leu251, Ala248, Ile261, Ile243, Leu246, Val256, and Leu264

Replacement amino acids will typically be amino acids with sidechains having similar size. For example, changes will often be: ile to arg, asp, asn, or lys; leu to pro, arg, or lys; val to asp, lys, or arg; and ala to lys.

### Example 6

### Target Residues for LPS Binding Protein Sequence

The sequence of a chimeric GP36CD-LPS Binding Protein is described in SEQ ID NO: 6. The residues which are indicated for replacement to generate a more soluble variant are:
Val248; Val267; Val269; Phe258; and Phe259.

### Example 7

### Soluble P271 (P266) Variant P317

As described above in Example 2, a soluble variant of the P271 protein was generated by substituting three different residues. The P317 variant incorporated different changes at two of the same locations. See SEQ ID NO: 7. P317 incorporated changes at V232 to K and V234 to K. As described above, the P271 was insoluble, while the P317 was soluble according to a solubility assay of sedimentation followed by PAGE.

### Example 8

### Native Human IL-13 Precurser

The sequence of the native human IL-13 precurser is provided as Accession number NP002179 and SEQ ID NO: 8. The sequence was entered into the TMHMM software with default parameters and provided:

TMHMM prediction
Sequence Length: 146

| | |
|---|---|
| # Sequence Number of predicted TMHs: | 1 |
| # Sequence Exp number of AAs in TMHs: | 36.85351 |
| # Sequence Exp number, first 60 AAs: | 22.67543 |
| # Sequence Total prob of N-in: | 0.79374 |

# Sequence POSSIBLE N-term signal sequence

| | | | | |
|---|---|---|---|---|
| Sequence | TMHMM2.0 | outside | 1 | 9 |
| Sequence | TMHMM2.0 | TMhelix | 10 | 32 |
| Sequence | TMHMM2.0 | inside | 33 | 146 |

The GRAVY software was applied to the segment from 1-32, based upon the TMHMM output, which calculated a Grand average of hydropathicity (GRAVY) from 1-32 amino acid region: 1.794. A DAS software analysis of this same region indicated:

The DAS curve for your query:
Potential transmembrane segments

| Start | Stop | Length | ~ | Cutoff |
|---|---|---|---|---|
| 8 | 27 | 20 | ~ | 1.7 |
| 9 | 25 | 17 | ~ | 2.2 |

The DAS curve showed peak about 4.4 at about residue 18 of the segment, predictive of a segment of high hydrophobicity. Based upon this information, locations for site directed mutagenesis (SDM) include those indicated in SEQ ID NO: 9, e.g., any of 9 modifications to the sequence. TMHMM analysis of this new sequence provided:

TMHMM prediction
Sequence Length: 146

| # Sequence Number of predicted TMHs: | | | 0 | |
|---|---|---|---|---|
| # Sequence Exp number of AAs in TMHs: | | | 10.40296 | |
| # Sequence Exp number, first 60 AAs: | | | 0.09921 | |
| # Sequence Total prob of N-in: | | | 0.08147 | |
| Sequence | TMHMM2.0 | outside | 1 | 146 |

The GRAVY software was applied to the new mutagenized segment from 1-32, as above, which calculated a Grand average of hydropathicity (GRAVY) 1-32 amino acid region: −0.312. A DAS software analysis of this same region indicated:

The DAS curve for your query:
Potential transmembrane segments

| Start | Stop | Length | ~ | Cutoff |
|---|---|---|---|---|
| 22 | 24 | 3 | ~ | 1.7 |

The DAS curve showed peak about 1.9 at about residue 23 of the segment. This suggests that the variant should be a soluble protein. This is confirmed using one or more of the analytical methods used to determine the solubility properties of a protein as described above. If desired, certain of the modifications incorporated may be removed to determine which combinations of modifications contribute most to change in solubility.

### Example 9

### Human BAX Protein

The sequence of human BAX protein is provided as Accession number Q07812 and SEQ ID NO: 10. The sequence was entered into the TMHMM software with default parameters and provided:

TMHMM prediction
Sequence Length: 192

| # Sequence Number of predicted TMHs: | | | 1 | |
|---|---|---|---|---|
| # Sequence Exp number of AAs in TMHs: | | | 20.77737 | |
| # Sequence Exp number, first 60 AAs: | | | 0.00139 | |
| # Sequence Total prob of N-in: | | | 0.12662 | |
| Sequence | TMHMM2.0 | outside | 1 | 168 |
| Sequence | TMHMM2.0 | TMhelix | 169 | 188 |
| Sequence | TMHMM2.0 | inside | 189 | 192 |

The GRAVY software was applied to the helix segment from 167-188, based upon the TMHMM output, which calculated a Grand average of hydropathicity (GRAVY) for the helix segment 167-188 sequence: 1.059. A DAS software analysis of the new sequence indicated:

The DAS curve for your query:
Potential transmembrane segments

| Start | Stop | Length | ~ | Cutoff |
|---|---|---|---|---|
| 8 | 17 | 10 | ~ | 1.7 |
| 9 | 16 | 8 | ~ | 2.2 |

The DAS curve showed peak about 2.8 at about residue 12 of the segment, corresponding to about residue 179 of the new sequence. Based upon this information, locations for site directed mutagenesis (SDM) include those indicated in SEQ ID NO: 11, e.g., any of 7 modifications to the sequence. TMHMM analysis of this new sequence provided:

TMHMM prediction
Sequence Length: 192

| | |
|---|---|
| # Sequence Number of predicted TMHs: | 0 |
| # Sequence Exp number of AAs in TMHs: | 0.5056 |
| # Sequence Exp number, first 60 AAs: | 0.00059 |
| # Sequence Total prob of N-in: | 0.05095 |

| | | | | |
|---|---|---|---|---|
| Sequence | TMHMM2.0 | outside | 1 | 192 |

The GRAVY software was applied to the new mutagenized sequence, as above, which calculated a Grand average of hydropathicity (GRAVY): −1.382. A DAS software analysis of the new sequence indicated:

The DAS curve for your query:
Potential transmembrane segments

| Start | Stop | Length | ~ | Cutoff |
|---|---|---|---|---|
| 12 | 13 | 2 | ~ | 1.7 |

The DAS curve showed peak of about 1.9 at about residue 12 of the segment, corresponding to about residue 179 of the new sequence. This suggests that the variant should be a soluble protein. This is confirmed using one or more of the analytical methods used to determine the solubility properties of a protein as described above. If desired, certain of the modifications incorporated may be removed to determine which combinations of modifications contribute most to change in solubility.

### Example 10

### Sec G, E. coli

The sequence of the Sec G protein from E. coli is provided as Accession number ZP12511033 and SEQ ID NO: 12. The sequence was entered into the TMHMM software with default parameters and provided:

TMHMM prediction
Sequence Length: 110

| | |
|---|---|
| # Sequence Number of predicted TMHs: | 2 |
| # Sequence Exp number of AAs in TMHs: | 41.2952 |
| # Sequence Exp number, first 60 AAs: | 28.96707 |
| # Sequence Total prob of N-in: | 0.99398 |

# Sequence POSSIBLE N-term signal sequence

| | | | | |
|---|---|---|---|---|
| Sequence | TMHMM2.0 | inside | 1 | 4 |
| Sequence | TMHMM2.0 | TMhelix | 5 | 22 |
| Sequence | TMHMM2.0 | outside | 23 | 50 |
| Sequence | TMHMM2.0 | TMhelix | 51 | 73 |
| Sequence | TMHMM2.0 | inside | 74 | 110 |

The GRAVY software was applied to the segment from 1-73, based upon the TMHMM output, which calculated a Grand average of hydropathicity (GRAVY) from 1-73 amino acid region: 1.279. A DAS software analysis of this 1-73 region indicated:

Potential transmembrane segments
The DAS curve for your query:
Potential transmembrane segments

| Start | Stop | Length | ~ | Cutoff |
|---|---|---|---|---|
| 5 | 20 | 16 | ~ | 2.2 |
| 5 | 21 | 17 | ~ | 1.7 |
| 57 | 70 | 14 | ~ | 1.7 |
| 58 | 69 | 12 | ~ | 2.2 |

The DAS curve showed peak about 5.8 at about residue 13 of the segment, corresponding to the same residue of the whole protein, second peak about 4.7 at about residue 65. Based upon this information, locations for site directed mutagenesis (SDM) include those indicated in SEQ ID NO: 13, e.g., any of 15 modifications to the sequence. TMHMM analysis of this new sequence provided:

TMHMM prediction
Sequence Length: 110

| | |
|---|---|
| # Sequence Number of predicted TMHs: | 0 |
| # Sequence Exp number of AAs in TMHs: | 8.80315 |
| # Sequence Exp number, first 60 AAs: | 8.80315 |
| # Sequence Total prob of N-in: | 0.07066 |

| | | | | |
|---|---|---|---|---|
| Sequence | TMHMM2.0 | outside | 1 | 110 |

The GRAVY software was applied to the new mutagenized segment from 1-73, as above, which calculated a Grand average of hydropathicity (GRAVY) 1-73 amino acid region: −0.278. A DAS software analysis of this same region indicated:

The DAS curve for your query:
Potential transmembrane segments

| Start | Stop | Length | ~ | Cutoff |
|---|---|---|---|---|
| 36 | 42 | 7 | ~ | 1.7 |

The DAS curve showed three peaks, peak below 1.5 at around residue 13 of the segment and the full protein; peak near 1.9 about residue 40; shoulder about 0.8 at around residue 58. This suggests that the variant should be a soluble protein. This is confirmed using one or more of the analytical methods used to determine the solubility properties of a protein as described above. If desired, certain of the modifications incorporated may be removed to determine which combinations of modifications contribute most to change in solubility.

### Example 11

### Kar2p Heat Shock Protein (BIP Homolog) Yarrowia

The sequence of the Yarrowia Kar2p heat shock protein is provided as Accession number Q99170 and SEQ ID NO: 14. The sequence was entered into the TMHMM software with default parameters and provided:

TMHMM prediction
Sequence Length: 670

| | |
|---|---|
| # Sequence Number of predicted TMHs: | 1 |
| # Sequence Exp number of AAs in TMHs: | 15.03465 |
| # Sequence Exp number, first 60 AAs: | 14.99038 |
| # Sequence Total prob of N-in: | 0.65486 |

# Sequence POSSIBLE N-term signal sequence

| | | | | |
|---|---|---|---|---|
| Sequence | TMHMM2.0 | inside | 1 | 6 |
| Sequence | TMHMM2.0 | TMhelix | 7 | 24 |
| Sequence | TMHMM2.0 | outside | 25 | 670 |

The GRAVY software was applied to the TMD portion segment from 7-24, based upon the TMHMM output, which calculated a Grand average of hydropathicity (GRAVY) for the TMD segment: 1.983. A DAS software analysis of the 7-24 segment indicated:

The DAS curve for your query:
Potential transmembrane segments

| Start | Stop | Length | ~ | Cutoff |
|---|---|---|---|---|
| 5 | 14 | 10 | ~ | 1.7 |
| 6 | 14 | 9 | ~ | 2.2 |

The DAS curve showed peak about 4 at about residue 11 of the segment, corresponding to about residue 18 of the complete sequence. Based upon this information, locations for site directed mutagenesis (SDM) include those indicated in SEQ ID NO: 15, e.g., any of 8 modifications to the sequence. TMHMM analysis of this new sequence provided:

TMHMM prediction
Sequence Length: 670

| # Sequence Number of predicted TMHs: | | | 0 | |
|---|---|---|---|---|
| # Sequence Exp number of AAs in TMHs: | | | 0.004650000000000000000000000000001 | |
| # Sequence Exp number, first 60 AAs: | | | 0.00267 | |
| # Sequence Total prob of N-in: | | | 0.00028 | |
| Sequence | TMHMM2.0 | outside | 1 | 670 |

The GRAVY software was applied to the new mutagenized segment from 7-24, as above, which calculated a Grand average of hydropathicity (GRAVY) 7-24 amino acid region: −1.328. A DAS software analysis of the new variant sequence indicated:

The DAS curve for your query:
Potential transmembrane segments

| | | | | |
|---|---|---|---|---|
| Start | Stop | Length | ~ | Cutoff |

[absence of prediction indicates low likelihood of transmembrane segment]

The DAS curve showed peak about 1.6 at about residue 11 of the segment, corresponding to about residue 18 of the whole sequence. This low peak suggests that the variant should be a soluble protein. This is confirmed using one or more of the analytical methods used to determine the solubility properties of a protein as described above. If desired, certain of the modifications incorporated may be removed to determine which combinations of modifications contribute most to change in solubility.

### Example 12

### Human Cathelecidin hCAP18

The sequence of the human cathelecidin hCAP18 (cathelidicin antimicrobial peptide preprotein) is provided as Accession number NP004336 and SEQ ID NO: 16. The sequence was entered into the TMHMM software with default parameters and provided:

TMHMM prediction
Sequence Length: 173

| | |
|---|---|
| # Sequence Number of predicted TMHs: | 1 |
| # Sequence Exp number of AAs in TMHs: | 22.55784 |
| # Sequence Exp number, first 60 AAs: | 22.55784 |
| # Sequence Total prob of N-in: | 0.95062 |

# Sequence POSSIBLE N-term signal sequence

| | | | | |
|---|---|---|---|---|
| Sequence | TMHMM2.0 | inside | 1 | 12 |
| Sequence | TMHMM2.0 | TMhelix | 13 | 35 |
| Sequence | TMHMM2.0 | outside | 36 | 173 |

The GRAVY software was applied to the segment from 13-35, based upon the TMHMM output, which calculated a Grand average of hydropathicity (GRAVY) from 13-35 amino acid region: 1.974, which is moderate hydrophobicity. A DAS software analysis of this same region indicated:

The DAS curve for your query:
Potential transmembrane segments

| Start | Stop | Length | ~ | Cutoff |
|---|---|---|---|---|
| 6 | 18 | 13 | ~ | 1.7 |
| 7 | 16 | 10 | ~ | 2.2 |

The DAS curve showed peak about 4.4 at about residue 11 of the segment, corresponding to about residue 24 of the full sequence. Based upon this information, locations for site directed mutagenesis (SDM) include those indicated in SEQ ID NO: 17, e.g., any of 5 modifications to the sequence. TMHMM analysis of this new sequence provided:

TMHMM prediction
Sequence Length: 173

| | |
|---|---|
| # Sequence Number of predicted TMHs: | 0 |
| # Sequence Exp number of AAs in TMHs: | 0.00038 |
| # Sequence Exp number, first 60 AAs: | 0.00038 |
| # Sequence Total prob of N-in: | 0.34024 |

| | | | | |
|---|---|---|---|---|
| Sequence | TMHMM2.0 | outside | 1 | 173 |

The GRAVY software was applied to the new mutagenized segment, as above, which calculated a Grand average of hydropathicity (GRAVY) 13-35 amino acid region: 0.161. A DAS software analysis of this same region indicated:

DAS prediction
[blank indicated absence of prediction; absence of prediction indicates low
likelihood of transmembrane segment]

The DAS curve showed peak about 0.9 at about residue 13 of the segment, corresponding to about residue 26 of the full sequence. The low peak of hydrophobicity and DAS prediction suggest that the variant should be a soluble protein. This is confirmed using one or more of the analytical methods used to determine the solubility properties of a protein as described above. If desired, certain of the modifications incorporated may be removed to determine which combinations of modifications contribute most to change in solubility.

### Example 13

### DNA Delivery Protein from Enterobacteria Phage PRD1

The sequence of the DNA delivery protein from enterobacteria phage PRD1 is provided as Accession number NP_{—040698} and SEQ ID NO: 18. The sequence was entered into the TMHMM software with default parameters and provided:

TMHMM prediction
Sequence Length: 207

| | |
|---|---|
| # Sequence Number of predicted TMHs: | 1 |
| # Sequence Exp number of AAs in TMHs: | 18.77386 |
| # Sequence Exp number, first 60 AAs: | 18.75108 |
| # Sequence Total prob of N-in: | 0.94833 |

# Sequence POSSIBLE N-term signal sequence

| | | | | |
|---|---|---|---|---|
| Sequence | TMHMM2.0 | inside | 1 | 12 |
| Sequence | TMHMM2.0 | TMhelix | 13 | 28 |
| Sequence | TMHMM2.0 | outside | 29 | 207 |

The GRAVY software was applied to the segment from 13-28, based upon the TMHMM output, which calculated a Grand average of hydropathicity (GRAVY) from 13-28 amino acid region: 2.237, which indicates a high hydrophobicity segment. A DAS software analysis of this same region indicated:

The DAS curve for your query:
Potential transmembrane segments

| | | | | |
|---|---|---|---|---|
| Start | Stop | Length | ~ | Cutoff |

[absence of prediction indicates low likelihood of transmembrane segment]

The DAS curve showed flat (broad) peak of about 1.5 at residues about 8-12 of the segment, corresponding to about residues 21-25 of the whole sequence. Based upon this information and results, locations for site directed mutagenesis (SDM) include those indicated in SEQ ID NO: 19, e.g., any of 4 modifications to the sequence. TMHMM analysis of this sequence provided:

TMHMM prediction
# Sequence Length: 207

| | |
|---|---|
| # Sequence Number of predicted TMHs: | 0 |
| # Sequence Exp number of AAs in TMHs: | 8.60369 |
| # Sequence Exp number, first 60 AAs: | 8.60107 |
| # Sequence Total prob of N-in: | 0.51615 |

| | | | | |
|---|---|---|---|---|
| Sequence | TMHMM2.0 | outside | 1 | 207 |

The GRAVY software was applied to the new mutagenized segment from 13-28, as above, which calculated a Grand average of hydropathicity (GRAVY) for the 13-28 amino acid region: −0.425, which indicates mild hydrophilicity of the segment. A DAS software analysis of this same region indicated:

DAS prediction
Potential transmembrane segments

| | | | | |
|---|---|---|---|---|
| Start | Stop | Length | ~ | Cutoff |

[absence of prediction indicates low likelihood of transmembrane segment]

The DAS curve showed flat peak about 1.4 at about residues 8-12 of the segment, corresponding to about residues 21-25 of the whole sequence. These suggest that the variant should be a soluble protein. This is confirmed using one or more of the analytical methods used to determine the solubility properties of a protein as described above. If desired, certain of the modifications incorporated may be removed to determine which combinations of modifications contribute most to change in solubility.

### Example 14

### Transglycosylase P7 from Enterobacteria Phage PRD1

The sequence of the transglycosylase P7 from enterobacteria phage PRD1 is provided as Accession number P27380 and SEQ ID NO: 20. The sequence was entered into the TMHMM software with default parameters and provided:

TMHMM prediction
Sequence Length: 265

| # Sequence Number of predicted TMHs: | | | 1 | |
|---|---|---|---|---|
| # Sequence Exp number of AAs in TMHs: | | | 28.96464 | |
| # Sequence Exp number, first 60 AAs: | | | 0.15622 | |
| # Sequence Total prob of N-in: | | | 0.39548 | |
| Sequence | TMHMM2.0 | outside | 1 | 216 |
| Sequence | TMHMM2.0 | TMhelix | 217 | 239 |
| Sequence | TMHMM2.0 | inside | 240 | 265 |

The GRAVY software was applied to the segment from 218-239, based upon the TMHMM output, which calculated a Grand average of hydropathicity (GRAVY) from 218-239 amino acid region: 2.559. A DAS software analysis of this same region indicated:

The DAS curve for your query:
Potential transmembrane segments

| Start | Stop | Length | ~ | Cutoff |
|---|---|---|---|---|
| 7 | 18 | 12 | ~ | 1.7 |
| 8 | 17 | 10 | ~ | 2.2 |

The DAS curve showed peak about 4.2 at about residue 12 of the segment, corresponding to about residue 230 of the whole sequence. Based upon this information, locations for site directed mutagenesis (SDM) include those indicated in SEQ ID NO: 21, e.g., any of 6 modifications to the sequence. TMHMM analysis of this new sequence provided:

TMHMM prediction
# Sequence Length: 265

| | |
|---|---|
| # Sequence Number of predicted TMHs: | 0 |
| # Sequence Exp number of AAs in TMHs: | 0.86067 |
| # Sequence Exp number, first 60 AAs: | 0.02158 |
| # Sequence Total prob of N-in: | 0.05164 |

| | | | | |
|---|---|---|---|---|
| Sequence | TMHMM2.0 | outside | 1 | 265 |

The GRAVY software was applied to the new mutagenized segment from 218-239, as above, which calculated a Grand average of hydropathicity (GRAVY) 218-239 amino acid region: 0.286, which is a low hydrophobicity measure. A DAS software analysis of this same region indicated:

The DAS curve for your query:
Potential transmembrane segments

| | | | | |
|---|---|---|---|---|
| Start | Stop | Length | ~ | Cutoff |

[absence of prediction indicates low likelihood of transmembrane segment]

The DAS curve showed peak about 1 at about residue 13 of the segment, corresponding to about residue 231 of the whole sequence. These suggest that the variant should be a soluble protein. This is confirmed using one or more of the analytical methods used to determine the solubility properties of a protein as described above. If desired, certain of the modifications incorporated may be removed to determine which combinations of modifications contribute most to change in solubility.

### Example 15

### Colicin N, Chain A, E. Coli

The sequence of the coli Chain A, Colicin N is provided as Accession number 1A87_A and SEQ ID NO: 22. The sequence was entered into the TMHMM software with default parameters and provided:

TMHMM prediction
Sequence Length: 321

| # Sequence Number of predicted TMHs: | | | 2 | |
|---|---|---|---|---|
| # Sequence Exp number of AAs in TMHs: | | | 42.75753 | |
| # Sequence Exp number, first 60 AAs: | | | 0.00011 | |
| # Sequence Total prob of N-in: | | | 0.48895 | |
| Sequence | TMHMM2.0 | outside | 1 | 256 |
| Sequence | TMHMM2.0 | TMhelix | 257 | 279 |
| Sequence | TMHMM2.0 | inside | 280 | 280 |
| Sequence | TMHMM2.0 | TMhelix | 281 | 303 |
| Sequence | TMHMM2.0 | outside | 304 | 321 |

The GRAVY software was applied to the segment from 258-303, based upon the TMHMM output, which calculated a Grand average of hydropathicity (GRAVY) for 259-303 amino acid region: −0.318. A DAS software analysis of this same region indicated:

The DAS curve for your query:
Potential transmembrane segments

| Start | Stop | Length | ~ | Cutoff |
|---|---|---|---|---|
| 8 | 39 | 32 | ~ | 1.7 |
| 10 | 22 | 13 | ~ | 2.2 |
| 29 | 37 | 9 | ~ | 2.2 |

The DAS curve showed broad peak about 2.8 at about residues 9-18 of the segment, corresponding to about residues 268-277 of the whole sequence; peak about 2.8 at about residue 36 of the segment, corresponding to about residue 295 of the whole sequence. Based upon these results, locations for site directed mutagenesis (SDM) include those indicated in SEQ ID NO: 23, e.g., any of 10 modifications to the sequence. TMHMM analysis of this new sequence provided:

TMHMM prediction
Sequence Length: 321

| | |
|---|---|
| # Sequence Number of predicted TMHs: | 0 |
| # Sequence Exp number of AAs in TMHs: | 0.00486 |
| # Sequence Exp number, first 60 AAs: | 0 |
| # Sequence Total prob of N-in: | 0.03166 |

| | | | | |
|---|---|---|---|---|
| Sequence | TMHMM2.0 | outside | 1 | 321 |

The GRAVY software was applied to the new mutagenized segment from 259-303, as above, which calculated a Grand average of hydropathicity (GRAVY) for 259-303 amino acid region: 0.008, which is neither hydrophobic nor hydrophilic. A DAS software analysis of this same region indicated:

The DAS curve for your query:
Potential transmembrane segments

| | | | | |
|---|---|---|---|---|
| Start | Stop | Length | ~ | Cutoff |

[absence of prediction indicates low likelihood of transmembrane segment]

The DAS curve showed broad peak about 1.3 at about residues 19-20 of the segment, corresponding to about residues 278-279 of the whole sequence. These results suggest that the variant should be a soluble protein. This is confirmed using one or more of the analytical methods used to determine the solubility properties of a protein as described above. If desired, certain of the modifications incorporated may be removed to determine which combinations of modifications contribute most to change in solubility.

### Example 16

### Colicin 1a, Chain A, E. Coli

The sequence of the E. coli Chain A, colicin 1a is provided as Accession number AAA59396 and SEQ ID NO: 24. The sequence was entered into the TMHMM software with default parameters and provided:

TMHMM prediction
Sequence Length: 602

| # Sequence Number of predicted TMHs: | | | 1 | |
|---|---|---|---|---|
| # Sequence Exp number of AAs in TMHs: | | | 25.36576 | |
| # Sequence Exp number, first 60 AAs: | | | 0 | |
| # Sequence Total prob of N-in: | | | 0.05593 | |
| Sequence | TMHMM2.0 | outside | 1 | 559 |
| Sequence | TMHMM2.0 | TMhelix | 560 | 582 |
| Sequence | TMHMM2.0 | inside | 583 | 602 |

The GRAVY software was applied to the segment from 561-582, based upon the TMHMM output, which calculated a Grand average of hydropathicity (GRAVY) for 561-582 amino acid region: 2.086. A DAS software analysis of this same region indicated:

The DAS curve for your query:
Potential transmembrane segments

| Start | Stop | Length | ~ | Cutoff |
|---|---|---|---|---|
| 9 | 13 | 5 | ~ | 1.7 |

The DAS curve showed peak about 2 at about residue 10 of the segment, corresponding to about residue 371 of the whole sequence. Based upon this information, locations for site directed mutagenesis (SDM) include those indicated in SEQ ID NO: 25, e.g., any of 7 modifications to the sequence. TMHMM analysis of this modified amino acid sequence provided:

TMHMM prediction
Sequence Length: 602

| | |
|---|---|
| # Sequence Number of predicted TMHs: | 0 |
| # Sequence Exp number of AAs in TMHs: | 0.00057 |
| # Sequence Exp number, first 60 AAs: | 0 |
| # Sequence Total prob of N-in: | 0.00097 |

| | | | | |
|---|---|---|---|---|
| Sequence | TMHMM2.0 | outside | 1 | 602 |

The GRAVY software was applied to the new mutagenized segment from 561-582, as above, which calculated a Grand average of hydropathicity (GRAVY) 561-582 amino acid region: −0.442, which is mildly hydrophilic. A DAS software analysis of this same region indicated:

The DAS curve for your query:
Potential transmembrane segments

| | | | | |
|---|---|---|---|---|
| Start | Stop | Length | ~ | Cutoff |

[absence of prediction indicates low likelihood of transmembrane segment]

The DAS curve showed peak about 1.5 at about residue 11 of the segment, corresponding to about residue 572. These suggest that the variant should be a soluble protein. This is confirmed using one or more of the analytical methods used to determine the solubility properties of a protein as described above. If desired, certain of the modifications incorporated may be removed to determine which combinations of modifications contribute most to change in solubility.

### Example 17

### Lambda Phage Holin

The sequence of the lambda phage holin is provided as Accession number

YP_{—001551775} and SEQ ID NO: 26. The sequence was entered into the TMHMM software with default parameters and provided:

TMHMM prediction
Sequence Length: 105

| | |
|---|---|
| # Sequence Number of predicted TMHs: | 2 |
| # Sequence Exp number of AAs in TMHs: | 53.228 |
| # Sequence Exp number, first 60 AAs: | 32.70055 |
| # Sequence Total prob of N-in: | 0.57409 |

# Sequence POSSIBLE N-term signal sequence

| | | | | |
|---|---|---|---|---|
| Sequence | TMHMM2.0 | inside | 1 | 6 |
| Sequence | TMHMM2.0 | TMhelix | 7 | 29 |
| Sequence | TMHMM2.0 | outside | 30 | 66 |
| Sequence | TMHMM2.0 | TMhelix | 67 | 89 |
| Sequence | TMHMM2.0 | inside | 90 | 105 |

The GRAVY software was applied to the segment from 8-89, based upon the TMHMM output, which calculated a Grand average of hydropathicity (GRAVY) for 8-89 amino acid segment: 0.992, which is moderate hydrophobicity. A DAS software analysis of this same region indicated:

The DAS curve for your query:
Potential transmembrane segments

| Start | Stop | Length | ~ | Cutoff |
|---|---|---|---|---|
| 14 | 20 | 7 | ~ | 1.7 |
| 17 | 18 | 2 | ~ | 2.2 |
| 40 | 49 | 10 | ~ | 1.7 |
| 43 | 46 | 4 | ~ | 2.2 |
| 64 | 72 | 9 | ~ | 1.7 |
| 67 | 70 | 4 | ~ | 2.2 |

The DAS curve showed peak about 2.2 at about residue 17 of the segment, corresponding to about residue 25 of the whole sequence; peak about 2.5 at about residue 47 of the segment, corresponding to about residue 55; peak about 2.4 at about residue 72 of the segment, corresponding to about residue 80. Based upon these results, locations for site directed mutagenesis (SDM) include those indicated in SEQ ID NO: 27, e.g., any of 10 modifications to the sequence, 2 of which are outside of the region of highest hydrophobicity. TMHMM analysis of this sequence provided:

TMHMM prediction
Sequence Length: 105

| | |
|---|---|
| # Sequence Number of predicted TMHs: | 0 |
| # Sequence Exp number of AAs in TMHs: | 0.03458 |
| # Sequence Exp number, first 60 AAs: | 0.02964 |
| # Sequence Total prob of N-in: | 0.51888 |

| | | | | |
|---|---|---|---|---|
| Sequence | TMHMM2.0 | outside | 1 | 105 |

The GRAVY software was applied to the new mutagenized segment from 8-89, as above, which calculated a Grand average of hydropathicity (GRAVY) for 8-89 amino acid region: −0.031, which is weakly hydrophilic. A DAS software analysis of this same region indicated:

The DAS curve for your query:
Potential transmembrane segments

| | | | | |
|---|---|---|---|---|
| Start | Stop | Length | ~ | Cutoff |

[absence of prediction indicates low likelihood of transmembrane segment]

The DAS curve showed peak of about 1.5 at residue 14 of the segment, corresponding to about residue 22 of the whole sequence; peak of about 1.3 at about residue 33 of the segment, corresponding to about residue 41; flat (broad) peak of about 1.2 at about residues 48-65 of the segment, corresponding to about residues 56-73. These are low hydrophobicity scores and suggest that the variant should be a soluble protein. This is confirmed using one or more of the analytical methods used to determine the solubility properties of a protein as described above. If desired, certain of the modifications incorporated may be removed to determine which combinations of modifications contribute most to change in solubility.

What is claimed is:

1. A method of identifying a variant protein of an insoluble first protein produced in a selected prokaryotic high expression system, said method comprising the steps of:
(i) selecting a first protein which is insoluble when produced in said selected prokaryotic high expression system;
(ii) identifying one or more residues in said protein which highly correlate with such insolubility; and
(ii) substituting said amino acid residue with a less hydrophobic amino acid residue;
thereby resulting in a variant protein which is recoverable in higher specific activity upon expression in said selected prokaryotic high expression system.

2. The method of claim 1, wherein said residues which highly correlate with such insolubility:
a) include highly hydrophobic residues in a segment of about 20 to 32 amino acids with a DAS score peak of at least about 2.3-2.5; or
b) are substituted with one or more amino acids with a hydrophobicity score at least about 0.5 less than said substituted residue.

3. The method of claim 1, wherein under said high expression system conditions said insoluble first protein forms inclusion bodies, while said variant protein does not form inclusion bodies when analogously expressed in the same prokaryotic high expression system.

4. The method of claim 1, wherein said:
a) residues which highly correlate with such insolubility include highly hydrophobic residues in a segment of about 19 to 31 amino acids with a transmembrane probability score of at least about 0.8 by TMHMM analysis;
b) one or more is at least three;
c) first protein is biologically active, and said variant protein has a higher specific activity in a crude lysate upon expression in said selected prokaryotic high expression system; or
d) first protein has 3 or fewer predicted transmembrane helices.

5. The method of claim 1, wherein said:
a) variant protein is expressed so that upon crude lysis harvest, said variant protein is in active form in an amount at least about 3-10 fold higher than said first protein;
b) less hydrophobic amino acid residue is an arginine, lysine, asparagine, glutamine, glutamic acid, or histidine; or
c) first protein has a DAS score on the predicted transmembrane helix of more than about 2.3.

6. The method of claim 1, wherein said:
a) prokaryote high expression system comprises either batch or fed batch growth periods;
b) variant protein has substantially the same number of residues as said first protein; or
c) first protein has a predicted transmembrane helix in the C terminus or middle portion.

7. The method of claim 1, wherein said:
a) a) residues include an isoleucine, valine, leucine, phenylalanine, cysteine, methionine, or alanine residue;
b) said prokaryote high expression system comprises a batch growth period; or
c) prokaryote high expression system comprises an inducible promoter.

8. The method of claim 1, wherein said:
a) residues include an isoleucine, valine, or leucine residue;
b) less hydrophobic amino acid residue is a proline, tyrosine, tryptophan, serine, or threonine;
c) first protein is less than about 300 amino acids; or
d) first protein has a predicted transmembrane helix in the N terminus portion or at the N terminus.

9. The method of claim 1, wherein said:
a) less hydrophobic amino acid residue is a hydrophilic amino acid residue;
b) variant protein is an enzyme; or
c) variant protein has at least 10× enzyme specific activity compared to said first protein in crude lysates when both are expressed in a similar high efficiency expression system.

10. The method of claim 1, wherein surface residue analysis is used to determine which residues which highly correlate with such insolubility are located at a location which interacts with the outer solvent, and a hydrophobic amino acid residue located at said location is substituted with a less hydrophobic residue.

11. The method of claim 10, wherein said:
a) variant has substantially the same number of residues as said first protein; or
b) first protein does not have a fusion tag or fusion protein attached.

12. The method of claim 10, wherein said variant protein is an enzyme.

13. A variant polypeptide of a first polypeptide which first polypeptide is insoluble upon high expression conditions in a prokaryotic expression host, said soluble variant:
a) containing one or more substitutions of a less hydrophobic amino acid residue at one or more positions of said first polypeptide within a region of about 19-33 contiguous residues exhibiting a peak DAS score of at least about 2.3-2.5; and
b) exhibiting a higher biological specific activity per weight of such polypeptide made than for said insoluble first polypeptide made in said prokaryotic expression host.

14. The variant polypeptide on claim 13, wherein said:
a) first polypeptide forms inclusion bodies in said high expression conditions; or
b) high expression conditions include a batch growth phase.

15. The variant polypeptide on claim 13, wherein said variant has:
a) a lower peak DAS score by at least about 0.3-0.5 than said first polypeptide; or
b) fewer than about 10% more residues than said first polypeptide.

16. The variant polypeptide on claim 13, wherein said variant has:
a) one or more is at least three; or
b) biological specific activity of the variant polypeptide during culture is at least about 3-7 fold greater than that of the first polypeptide.

17. A variant protein of a first protein possessing a segment of about 20 to 35 amino acids which TMHMM analysis provides a transmembrane probability of at least about 0.7 and is insoluble upon high expression conditions in a prokaryotic expression host, said soluble variant protein:
a) containing one or more substitutions of a less hydrophobic amino acid residue at one or more positions in said segment of said first protein; and
b) exhibiting a higher biological specific activity per weight of such protein made than for said insoluble first protein made in said prokaryotic expression host.

18. The variant protein of claim 17, wherein a corresponding segment or said variant protein to said segment of at least 20 amino acids possessed by said first protein has a transmembrane probability score of less than 0.5.

19. The variant protein of claim 17, wherein said:
a) substitutions of a less hydrophobic amino acid residue include arginine, lysine, asparagines, aspartic acid, glutamine, glutamic acid, or histidine; or
b) variant protein can provide about 2-5 times more units of soluble biological activity per gram of cells than said first protein when both are produced in said high expression system conditions.
